SEQUENCE LISTING (3 sequences)

SEQ ID NO: 1 - protein, 7 aa, Artificial Sequence
  Description of Artificial Sequence Synthetic peptide
EASGGPE

SEQ ID NO: 2 - protein, 4 aa, Artificial Sequence
  Description of Artificial Sequence Synthetic peptide
KDEL

SEQ ID NO: 3 - protein, 5 aa, Pseudomonas aeruginosa
REDLK

(19) United States
(12) Patent Application Publication
(10) Pub. No.: US 2019/0388521 A1
(43) Pub. Date: Dec. 26, 2019

(54) REDUCTION OF EGFR THERAPEUTIC TOXICITY

(71) Applicant: REGENTS OF THE UNIVERSITY OF MINNESOTA, Minneapolis, MN (US)

(72) Inventors: Jaime F. Modiano, Minneapolis, MN (US); Daniel Vallera, Minneapolis, MN (US); Antonella Borgatti, Minneapolis, MN (US)

(21) Appl. No.: 16/404,071

(22) Filed: May 6, 2019

Related U.S. Application Data
(63) Continuation of application No. 15/280,673, filed on Sep. 29, 2016, now abandoned.
(60) Provisional application No. 62/235,314, filed on Sep. 30, 2015.

(51) Int. Cl. A61K 38/48; A61K 9/00; A61K 38/18; A61K 38/16

(52) U.S. Cl. CPC A61K 38/482; A61K 9/0019; A61K 38/164; A61K 38/1808; C12Y 304/21073

(57) ABSTRACT

In certain embodiments, the present invention provides a method of treating a subject having a tumor that expresses EGFR and/or uPAR, even if at low levels. In certain embodiments, the present invention provides a method of preventing hemangiosarcoma (HSA) in a dog predisposed to developing HSA or angiosarcoma in a human predisposed to developing angiosarcoma. In certain embodiments, the present invention provides a method of preventing a hemangiosarcoma (HSA) in a dog that is positive for HSA by means of a blood test but negative by tumor imaging.

### RELATED APPLICATIONS

This is a continuation application of U.S. patent application Ser. No. 15/280,673, pending, filed Sep. 29, 2016, which claims priority to U.S. Provisional Application No. 62/235,314 that was filed on Sep. 30, 2015, the disclosures of which are incorporated herein by reference thereto.

### U.S. GOVERNMENT RIGHTS

This invention was made with government support under CA36725, CA082154, and OD017242 awarded by the National Institutes of Health. The government has certain rights in the invention.

### SEQUENCE LISTING

This application contains a Sequence Listing electronically submitted via EFS-Web to the United States Patent and Trademark Office as an ASCII text file entitled “0110000574_SEQUENCELISTING_ST25.txt” having a size of 9 kilobytes and created on Nov. 18, 2016. The information contained in the Sequence Listing is incorporated by reference herein.

### BACKGROUND OF THE INVENTION

Sarcomas comprise a heterogeneous group of rare malignancies from mesenchymal tissues accounting for 1% to 3% of all human tumors. Of the estimated 1.6M tumors diagnosed in the United States in 2014, approximately 12,000 were soft tissue sarcomas, and an additional 3,000 were tumors of bones and joints The incidence of sarcomas is very similar in Europe (Cassier et al, Ann Oncol. 2014 June; 25(6):1222-8) and the rest of the developed world. Despite widespread activity in the area of targeted therapies for sarcomas (Frith et al, Curr Oncol Rep. 2013 August; 15(4):378-85), the relatively small number of cases diagnosed each year and the extensive heterogeneity of these tumors has made progress challenging. In fact, outside of Imatinib, which is used to target mutant c-Kit in gastrointestinal stromal tumors (GIST), there are no approved or generally accepted, safe and effective targeted therapies for sarcomas. Furthermore, contemporary clinical trials with small molecules used to target tyrosine kinases and other signaling molecules have shown very poor objective response rates (below 5%) and clinical benefit rates (generally below 30%) for sarcomas (Chmielowski et al, Expert Rev Anticancer Ther. 2012 September; 12(9):1217-28).

In contrast to their infrequent occurrence in humans, spontaneous sarcomas of soft tissue and bone are diagnosed very commonly in companion dogs (see Fenger et al, ILAR J. 2014; 55(1):69-85). Canine hemangiosarcoma (HSA) is one of the most common, most aggressive, and least curable spontaneous sarcomas of dogs. These tumors arise from cells that line blood vessels. Of the approximately 65 million owned dogs in the United States in 2004, between 1.5 and 2.5 million will get this disease and die from it. The disease accounts for about 7% of all canine cancers. Because the disease is extremely indolent, treatment is largely ineffective and microscopic metastases are often present at the time of diagnosis. The tumors at this stage are largely resistant to chemotherapy, and thus standard-of-care (surgery and intensive chemotherapy) provides a median survival of little more than six months (Clifford, C. A., et al. (2000) J. Vet. Intern. Med. 14:479-485; Sorenmo, K., et al. (2000), J. Vet. Intern. Med. 14:395-398; and Sorenmo, K. U., et al. (1993) J. Vet. Intern. Med. 7:370-376). Common primary sites for HSA are spleen and right atrium (visceral), and subcutis. Local infiltration and systemic metastases are the common growth patterns and metastatic sites are wide spread, with lung and liver being the most frequently affected organs (Oksanen, A. (1978) J. Comp. Pathol. 88:585-595; and Brown, N. O., et al., (1985) J. Am. Vet. Med. Assoc. 186:56-58). Morbidity and mortality are usually due to acute internal hemorrhage secondary to tumor rupture. Many dogs die from severe abdominal or thoracic hemorrhage before any treatment can be instituted. Although dogs of any age and breed are susceptible to HSA, it occurs more commonly in dogs beyond middle age, and in breeds such as Golden Retrievers, German Shepherd Dogs, Portuguese Water Dogs, and Skye Terriers, among others. The estimated lifetime risk of HSA in Golden Retrievers is 1 in 5, illustrating the magnitude of this problem.

Human angiosarcomas are similar to canine HSA (see, e.g., Fosmire, S. P., et al (2004) Laboratory Investigation 84:562-572). These tumors are uncommon soft tissue sarcomas that can arise in a variety of locations, such as the liver, spleen, skin breast and endocrine organs (see, e.g., Fedok, F. G., et al. (1999) Am J. Otolaryngol. 20:223-231; Hai, S. A., et al., (2000) J. Natl. Med. Assoc. 92:143-146; and Budd, G. T. (2002) Curr. Oncol. Rep. 4:515-519). Like canine HSA, treatment of human angiosarcomas can be challenging and often is not successful.

In particular, with the exception noted above where c-Kit-mutant GIST can be targeted with Imatinib, implementation of targeted therapies for sarcomas has suffered from the lack of recurrent mutations or recurrent overexpression of proteins that can be targeted therapeutically with currently available molecules (Frith et al, Curr Oncol Rep. 2013 August; 15(4):378-85 4; Linch, Nat Rev Clin Oncol. 2014 April; 11(4):187-202). The absence of such targets eliminates selectivity, such that therapies targeting proteins which are expressed physiologically in vital organs and tissues, such as skin, liver, gut, kidney, brain, and heart, including but not limited to epidermal growth factor receptors (EGFRs), vascular endothelial growth factor receptors (VEGFRs), urokinase receptors (uPARs), mammalian target of rapamycin (mTOR), etc., are bound to have unacceptable toxicity, including fatal adverse events. Such toxicities are observed even in patients with tumors that overexpress targets, as is seen in patients with lung cancer, head and neck cancer, breast cancer, and others treated with tyrosine kinase inhibitors or antibodies directed against epidermal growth factor receptors (EGFRs), vascular endothelial growth factor receptors (VEGFRs), and other cancer targets (Funakoshi et al, Cancer Treat Rev. 2014 June; 40(5):636-47; Launay-Vacher, Ann Oncol. 2015 August; 26(8): 1677-84).

The poor outcomes of human sarcoma patients, as well as of dogs and other companion animals with sarcomas, and the paucity of treatments for these cancers are significant unmet needs in contemporary medicine and veterinary medicine.

### SUMMARY OF THE INVENTION

In certain embodiments, the present invention provides a method of treating a subject having a tumor that expresses EGFR and/or uPAR, even if at low levels, comprising:

(a) administering intravenous fluids to the subject at a rate of about 0.1 to 1 ml/kg/hr for about 10 to 60 minutes, and

(b) systemically administering a therapeutic composition at a dosage regime of 25 to 100 μg/kg, wherein the therapeutic composition is administered three times in a one-week period.

In certain embodiments, the present invention provides a method of treating a subject having a tumor that expresses EGFR and/or uPAR, even if at low levels, that would otherwise be at risk for toxicity related to EGFR and or uPAR targeted therapies, comprising:

(a) administering intravenous fluids to the subject at a rate of about 0.1 to 1 ml/kg/hr for about 10 to 60 minutes, and

(b) systemically administering a therapeutic composition at a dosage regime of 25 to 100 μg/kg, wherein the therapeutic composition is administered three times in a one-week period.

In certain embodiments, the present invention provides a method of preventing hemangiosarcoma (HSA) in a dog predisposed to developing HSA or angiosarcoma in a human predisposed to developing angiosarcoma comprising:

(a) administering intravenous fluids to the subject at a rate of about 0.1 to 1 ml/kg/hr for about 10 to 60 minutes, and (b) systemically administering a therapeutic composition at a dosage regime of 25 to 100 μg/kg, wherein the therapeutic composition is administered three times in a one-week period.

In certain embodiments, the present invention provides a method of preventing a hemangiosarcoma (HSA) in a dog that is positive for HSA by means of a blood test but negative by tumor imaging comprising: (a) administering intravenous fluids to the subject at a rate of about 0.1 to 1 ml/kg/hr for about 10 to 60 minutes, and (b) systemically administering a therapeutic composition at a dosage regime of 25 to 100 μg/kg, wherein the therapeutic composition is administered three times in a one-week period. In certain embodiments the blood test comprises: (a) providing a population of cells obtained from a blood sample from the dog; (b) determining (i) the level at which cells within the cell population concurrently express a plurality of cell markers, the plurality of cell markers comprising at least one primitive hematopoietic cell marker and at least one endothelial cell marker, and (ii) whether or not cells within the cell population express at least one leukocyte-specific cell marker, wherein the at least one primitive hematopoietic cell marker is selected from the group consisting of CD117, CD34, and CD133; the at least one endothelial cell marker is selected from the group consisting of CD51/CD61, CD31, CD105, CD106 CD146 and von Willebrand Factor (vWF);

and the at least one leukocyte-specific cell marker is selected from the group consisting of CD18, CD3, CD5, CD21 and CD11b; and (c) comparing the level at which cells in the cell population concurrently express the plurality of cell markers with a control level of concurrent expression of the markers, wherein (1) an increase in the expression level of the plurality of cell markers relative to the control expression level, and (2) the absence of expression of CD18, CD3, CD5, CD21 and/or CD11b collectively are an indication of hemangiosarcoma (See, e.g., U.S. Pat. No. 7,910,315).

In certain embodiments, the present invention provides a method of preventing angiosarcoma in a human that is positive for angiosarcoma by means of a blood test but negative by tumor imaging comprising: (a) administering intravenous fluids to the subject at a rate of about 0.1 to 1 ml/kg/hr for about 10 to 60 minutes, and (b) systemically administering a therapeutic composition at a dosage regime of 25 to 100 μg/kg, wherein the therapeutic composition is administered three times in a one-week period.

In certain embodiments, the level of EGFR is a low level of EGFR (for example, less than 10 fragments per kilobase per million reads in RNA sequencing or <1% of positive cells labeled by immunohistochemistry in a tumor).

In certain embodiments, the level of uPAR is a low level of uPAR (for example, less than 100 fragments per kilobase per million reads in RNA sequencing or <3% of positive cells labeled by immunohistochemistry in a tumor).

In certain embodiments, the subject has a tumor that expresses low levels of EGFR and/or uPAR that would otherwise be at risk for toxicity related to EGFR and or uPAR targeted therapies.

In certain embodiments, the tumor is a carcinoma or a sarcoma. (Linch, Nat Rev Clin Oncol. 2014 April; 11(4):187-202 (sarcomas); Frith et al, Curr Oncol Rep. 2013 August; 15(4):378-85 (sarcomas); Funakoshi et al, Cancer Treat Rev. 2014 June; 40(5):636-47 (carcinomas and others).

In certain embodiments, the sarcoma is a hemangiosarcoma (HSA) or an angiosarcoma. In certain embodiments, the subject is a human and the tumor is a sarcoma.

In certain embodiments, the tumor is leiomyosarcoma, liposarcoma, undifferentiated sarcoma, synovial sarcoma, MPNST, MFH, mixosarcoma, myxofibrosarcoma, rhabdosarcoma, osteosarcoma, and/or ewing sarcoma.

In certain embodiments, the subject is a dog and the tumor is an HSA.

In certain embodiments, the subject is a dog and the tumor is a soft tissue sarcoma.

In certain embodiments, the subject is a dog and the tumor is a carcinoma or a melanoma.

In certain embodiments, step (b) is repeated one or more times.

In certain embodiments, the method further comprises (c) administering chemotherapy.

In certain embodiments, the method further comprises (d) repeating step (b).

In certain embodiments, the therapeutic composition is a bispecific EGF angiotoxin (“eBAT”, also called “BEAT”), wherein eBAT is EGF. In certain embodiments, the therapeutic composition comprises EGFATFKDEL mut7.

In certain embodiments, the therapeutic composition comprises EGFKDEL and ATFKDEL, wherein EGFKDEL and ATFKDEL are administered separately, simultaneously or sequentially.

In certain embodiments, the dosage of the therapeutic composition is about 50 μg/kg.

In certain embodiments, the therapeutic composition is administered by means of a slow IV push. In certain embodiments, the product is drawn into a syringe and the syringe is connected to a pump that can deliver the volume of drug through an indwelling catheter over a desired time-point, which can extend from 20 to 180 minutes or longer.

In certain embodiments, steps (a) and (b) are repeated about one week after initial treatment. In certain embodiments, steps (a) and (b) are repeated about 21 days after the original administration. In certain embodiments, steps (a) and (b) are repeated again about 62 days later (about 9 weeks).

In certain embodiments, the systemic administration is by means of intravenous, intraperitoneal or subcutaneous administration.

In certain embodiments, the administration the administration is for a duration of at least six months.

### BRIEF DESCRIPTION OF THE FIGURES

FIGS. 1A-1E. Construction and in vitro activity of eBAT: Bispecific eBAT was studied for its activity against canine and human sarcoma cells. A) Expression vector for eBAT, human EGF and the high affinity amino terminal fragment of urokinase linked to a deimmunized PE₃₈KDEL molecule. The fusion gene (from 5′ end to 3′ end) consisted of an Ncol restriction site, the genes for human EGF, an ATG initiation codon, the downstream 135-amino terminal fragment (ATF) from uPA linked by a 20 amino-acid segment of human muscle aldolase (HMA), the 7 amino-acid EASGGPE (SEQ ID NO:1) linker, the first 362 amino acids of the pseudomonas exotoxin (PE) molecule with KDEL (SEQ ID NO:2) at the C terminus, and a NotII restriction site at the 3′ end of the construct. B) Canine EMMA cells were treated with various concentrations of eBAT (EGFATFKDEL) and control CD3CD3KDEL and then protein synthesis was measured 3 days later using a tritiated leucine uptake assay. C) Human U-20S Osteosarcoma cells were treated with various concentrations of eBAT tested against EGF4KDEL and then proliferation was measured 3 days later using a tritiated thymidine uptake assay. D) Human TC-71 Ewing's sarcoma cells were treated with various concentrations of eBAT tested against EGF4KDEL and CD19KDEL as negative control. Proliferation was measured 3 days later using a tritiated thymidine uptake assay. E) eBAT is tested against HPB-MLT cells to test specificity. eBAT, EGF4KDEL and 2219KDEL showed no significant cytotoxicity.

FIGS. 2A-2F. EGFR and PLAUR gene expression analysis in human sarcomas and spontaneous canine tumors. (A) EGFR and PLAUR gene expression analysis was done in 212 tumor tissue samples extracted from the TCGA database. The X-axis represents the patients supervised by tumor type and the Y-axis is the expression intensity as fragments per kilobase of transcript per million (FPKM) mapped reads. (B) Unsupervised hierarchical cluster and heat map highlighting EGFR and PLAUR expression in the human TCGA dataset. (C) EGFR and uPAR protein expression is shown in TMAs constructed from human synovial sarcoma tissue samples. The X-axis represents patient TMAs and the Y-axis represents optical density of EGFR and uPAR on immunohistochemistry. D) EGFR and PLAUR gene expression analysis in an independent dataset of canine hemangiosarcoma samples. (E) EGFR and PLAUR gene expression analysis in canine osteosarcoma samples. (F) EGFR and PLAUR gene expression analysis in canine lymphoma samples. Tumor-bearing dogs are on the X-axis and fragments per kilobase of transcript per million mapped reads on the Y-axis, illustrating the levels of EGFR and PLAUR expression from the individual tumors. The following detailed values pertain to gene expression in TCGA samples of EGFR and PLAUR, respectively: Count: 212, 212; Mean (FPKM):653.4, 1,713; Mean (FPKM) lower confidence limit: 548.7, 1,387; Mean (FPKM) upper confidence limit: 758.0, 2,040; Variance: 600,273, 5,844,287; Standard Deviation: 774.8, 2,418; Mean Standard Error: 53.1, 165; Coefficient of Variation: 1.2, 1.4; Minimum (FPKM): 3.1, 40.9; Minimum (FPKM): 6,575.1, 19,171.7; Median (FPKM): 410.0, 757.9; Median Error: 4.56, 14.2; Percentile 25%(Q1): 215.4, 250.4; Percentile 75% (Q3): 752.9, 2,149.

FIGS. 3A-3F and 4A-4D. EGFR and uPAR expression in human synovial sarcomas and canine HSA TMA from 15 dogs in the SRCBST study. Synovial cell sarcoma TMA spots immunohistochemically stained for EGFR and uPAR. Representative highly and lowly stained spots for EGFR are shown (3A-3B human) (4A-4B canine). Note intense staining in mononuclear and inflammatory cells in panel 4A. Representative highly and lowly stained spots for uPAR are shown (3C-3D human) (4C-4D canine). An example of heterogeneous expression of uPAR is shown in the human synovial TMA where uPAR expression is much higher in the glandular cells staining darkly brown and forming elongated glands, sometimes with compressed slit-like spaces between the gland cells (3E). An admixture of spindled and glandular cells imparting a marbled-like appearance is also shown (3F).

FIGS. 5A-5B. Effect of eBAT on survival of dogs with splenic HSA treated with adjuvant doxorubicin chemotherapy. (A) Kaplan-Meier Curve for all 23 dogs in the SRCBST-1 study versus the comparison dogs. (B) Kaplan-Meier Curve for the 17 dogs treated at the biologically active dose versus the comparison dogs. Curves illustrate prolongation of survival in dogs treated with eBAT compared to the comparison group.

FIG. 6. Survival probability based on EGFR expression. Graph illustrating death events in patients where EGFR expression was in the upper 50^{th} percentile versus patients with EGFR expression in the lower 50^{th} percentile.

FIGS. 7A-7F. Correlation plots between EGFR and PLAUR (uPAR). EGFR and PLAUR expression was examined as described in the Materials and Methods section in human tumors from the TCGA database, TMAs constructed from human synovial sarcoma tissue samples, and datasets of canine HSA, canine osteosarcoma, and canine lymphoma. EGFR expression is plotted on the Y-axis and PLAUR/uPAR expression on the X-axis.

FIG. 8. Enrollments, exclusions, and assessments. Flow chart with details of dogs enrolled in the study and exclusions from each of the measured endpoints.

FIG. 9. Flow-steps followed to treat dogs at risk or humans at risk because they have tested positive for circulating tumor cells.

### DETAILED DESCRIPTION OF THE INVENTION

### Definitions

As used in this specification and the appended claims, the singular forms “a,” “an” and “the” include plural references unless the content clearly dictates otherwise.

Unless defined otherwise, all technical and scientific terms used herein have the meaning commonly understood by a person skilled in the art to which this invention belongs. The following references provide one of skill with a general definition of many of the terms used in this invention: Stedman, T. L., STEDMAN'S MEDICAL DICTIONARY (26th ed., 1995); Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991).

The term “sarcoma” has its normal meaning in the art and refers generally to a malignant tumor of connective or other nonepithelial tissue.

The term “carcinoma” has its normal meaning in the art and refers generally to is a type of cancer that develops from epithelial cells. Specifically, a carcinoma is a cancer that begins in a tissue that lines the inner or outer surfaces of the body, and that generally arises from cells originating in the endodermal or ectodermal germ layer during embryogenesis.

The term “hemangiosarcoma” has its normal meaning in the art and refers generally to malignant neoplasms that are characterized by rapidly proliferating, extensively infiltrating, anaplastic cells derived from blood vessels and lining irregular blood-filled or lumpy spaces.

“Angiosarcoma” as used herein has its normal meaning in the art and refers generally to malignant neoplasms occurring most often in the liver, spleen, skin, breast and endocrine organs.

The term “leukemia” has its normal meaning in the art and generally refers to a disease involving the progressive proliferation of abnormal leukocytes found in hematopoietic tissues, other organs, and usually in the blood in increased numbers. Symptoms of the disease typically include severe anemia, hemorrhages, and enlargement of lymph nodes or the spleen.

Lymphoma” as used herein refers generally to cancers that develop in the lymphatic system. In humans, one specific type of lymphoma is called Hodgkin's disease, which can be endemic (caused by Epstein Barr virus-dependent transformation of B lymphocytes) or sporadic (not associated with Epstein Barr virus infection), and is characterized by the presence of Reed Sternberg cells. All other lymphomas are grouped together and are called non-Hodgkin's lymphomas.

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like.

As used herein, references to specific polypeptides (e.g., cell markers such as CD117, CD34, CD51/61, CD18, CD45, CD31, CD105 and CD146) refer to a polypeptide having a native amino acid sequence, as well naturally occurring variant forms (e.g., alternatively spliced forms), naturally occurring allelic variants and forms including postranslational modifications. As noted above, the specific protein markers referred to herein include the protein as expressed in various mammals, including humans and dogs.

The term “antibody” as used herein includes, but is not limited to, antibodies obtained from both polyclonal and monoclonal preparations, as well as the following: (i) chimeric antibody molecules (see, for example, Winter et al. (1991) Nature 349:293-299; and U.S. Pat. No. 4,816,567); (ii) F(ab′)2 and F(ab) fragments; (iii) Fv molecules (noncovalent heterodimers, see, for example, Inbar et al. (1972) Proc. Natl. Acad. Sci. USA 69:2659-2662; and Ehrlich et al. (1980) Biochem 19:4091-4096); (iv) single-chain Fv molecules (sFv) (see, for example, Huston et al. (1988) Proc. Natl. Acad. Sci. USA 85:5879-5883); (v) dimeric and trimeric antibody fragment constructs; (vi) humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); (vii) Mini-antibodies or minibodies (i.e., sFv polypeptide chains that include oligomerization domains at their C-termini, separated from the sFv by a hinge region; see, e.g., Pack et al. (1992) Biochem 31:1579-1584; Cumber et al. (1992) J. Immunology 149B:120-126); and, (vii) any functional fragments obtained from such molecules, wherein such fragments retain specific-binding properties of the parent antibody molecule.

The phrases “specifically binds” when referring to a protein, “specifically immunologically cross reactive with,” or simply “specifically immunoreactive with” when referring to an antibody, refers to a binding reaction which is determinative of the presence of the protein in the presence of a heterogeneous population of proteins and other biologics. Thus, under designated conditions, a specified ligand binds preferentially to a particular protein and does not bind in a significant amount to other proteins present in the sample. A molecule or ligand (e.g., an antibody) that specifically binds to a protein has an association constant of at least 10³M⁻¹ or 10⁴M⁻¹, sometimes 10⁵ M⁻¹ or 10⁶M⁻¹, in other instances 10⁶M⁻¹ or 10⁷M⁻¹, preferably 10⁸M⁻¹ to 10⁹M⁻¹, and more preferably, about 10¹⁰ M⁻¹ to 10¹¹M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

The term “label” refers generally to an agent that can be detected by some means (e.g., chemical, physical, electromagnetic or other analytical means). Examples of detectable labels that can be utilized include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

A “subject” can be a mammal, including primates, non-human primates (e.g., monkey, ape, chimpanzee) and mammals other than primates (e.g., cat, dog, rat, mouse). Most typically the subject is a human or a dog.

A difference is typically considered to be “statistically significant” in general terms if an observed value differs by more than the level of experimental error. A difference, for example, can be “statistically significant” if the probability of the observed difference occurring by chance (the p-value) is less than some predetermined level. As used herein a “statistically significant difference” refers to a p-value that is <0.05, preferably <0.01 and most preferably <0.001.

A “control value” or simply “control” generally refers to a value (or range of values), such as expression levels, against which an experimental or determined value is compared. As used herein, the term typically refers to a measure of expression of one or more markers in a sample from a particular individual or population of individuals. For instance, the term can refer to the concentration of cells expressing one or more markers (e.g., the concentration of cells having a particular expression profile) in a sample. In the case of methods in which the risk of hemangiosarcoma or angiosarcoma is being evaluated, the control is typically the concentration or frequency of cells from the same tissue or body fluid as those under test having a particular expression profile as determined for an individual or population of individuals at low-risk for the disease and/or that has no discernible evidence of the disease (e.g., no detectable clinical manifestations). The control can also be the test sample analyzed with an irrelevant antibody or probe or primer instead of an antibody, probe or primer to a desired marker. If the signal from the antibody, probe or primer to the desired marker is not higher than that of the irrelevant control (and a margin of experimental error) expression is considered to be absent. Conversely, if the signal from the antibody, primer or probe to the desired marker is higher than that from an irrelevant control and an appropriate margin of experimental error, the marker is expressed. For comparison of leukemia cell marker levels, test samples can be compared with samples from the same tissue or body source either with individuals at low risk of disease (hemangiosarcoma or leukemia) or individuals known to have leukemia. Examples of suitable controls for dogs include those at low risk for hemangiosarcoma, i.e., dogs other than those at high risk (e.g., dogs beyond middle age, Golden Retrievers, German Shepherd Dog, Portuguese Water Dogs, Skye Terriers, or mixed breed dogs containing predominant derivation from such breeds). Absence of clinical manifestation of hemangiosarcoma or angiosarcoma can be evaluated by imaging techniques such as ultrasound, radiographs and/or magnetic imaging techniques (e.g., MRI), for instance. The control can be based upon a single individual, but more typically is a statistical value (e.g., an average or mean) determined from a population. The control can be determined contemporaneously with the test or experimental value or can be performed prior to the test assay. Thus, the control can be based upon contemporaneous or historical data.

In some methods, the control is a “threshold level.” A “threshold level” as used herein generally refers to a threshold value for the expression level of one or more markers that are associated with hemangiosarcoma and/or angiosarcoma. In some instances, the threshold level is expressed as the concentration of cells that concurrently express the one or more markers of interest. If a measured value for the expression level of the markers in a test sample is above the threshold level, this is a statistically-significant indication that the test sample is from a subject that has hemangiosarcoma or angiosarcoma. If, however, the measured value of the test sample is below the threshold level, this is a statistically significant indication that the test sample is from a subject that does not have hemangiosarcoma or angiosarcoma. As with control values, a threshold level can be based upon a single individual, but more commonly represents a value determined from a population of samples to provide the desired level of statistical certainty. Thus, the threshold value is often a statistical value (e.g., an average or mean) established for a population of individuals.

The terms “nucleic acid,” “polynucleotide,” and “oligonucleotide” are used herein to include a polymeric form of nucleotides of any length, including, but not limited to, ribonucleotides or deoxyribonucleotides. There is no intended distinction in length between these terms. Further, these terms refer only to the primary structure of the molecule. Thus, in certain embodiments these terms can include triple-, double- and single-stranded DNA, as well as triple, double- and single-stranded RNA. They also include modifications, such as by methylation and/or by capping, and unmodified forms of the polynucleotide. More particularly, these terms include polymers containing nonnucleotidic backbones, for example, polyamide (e.g., peptide nucleic acids (PNAs)) and polymorpholino polymers, and other synthetic sequence-specific nucleic acid polymers, providing that the polymers contain nucleobases in a configuration which allows for base pairing and base stacking, such as is found in DNA and RNA.

The term “expression” or “express” refers to the conversion of sequence information, contained in a gene, into a gene product. The gene product can be the direct transcriptional product of a gene (e.g., a mRNA) or a protein produced by translation of a mRNA. Gene products also include RNAs that are modified, by processes such as capping, polyadenylation, methylation, and editing, and proteins modified by, for example, methylation, acetylation, phosphorylation, ubiquitination, ADP-ribosylation, and glycosylation.

A “probe” is a nucleic acid capable of binding to a target nucleic acid of complementary sequence through one or more types of chemical bonds, usually through complementary base pairing, usually through hydrogen bond formation, thus forming a duplex structure. The probe binds or hybridizes to a “probe binding site.” The probe can be labeled with a detectable label to permit facile detection of the probe, particularly once the probe has hybridized to its complementary target. The label attached to the probe can include any of a variety of different labels known in the art that can be detected by chemical or physical means, for example. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates. Probes can vary significantly in size. Some probes are relatively short. Generally, probes are at least 7 to 15 nucleotides in length. Other probes are at least 20, 30 or 40 nucleotides long. Still other probes are somewhat longer, being at least 50, 60, 70, 80, 90 nucleotides long. Yet other probes are longer still, and are at least 100, 150, 200 or more nucleotides long. Probes can be of any specific length that falls within the foregoing ranges as well.

A “primer” is a single-stranded polynucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically is at least 7 nucleotides long and, more typically range from 10 to 30 nucleotides in length. Other primers can be somewhat longer such as 30 to 50 nucleotides long. Short primer molecules generally require cooler temperatures to form sufficiently stable hybrid complexes with the template. A primer need not reflect the exact sequence of the template but must be sufficiently complementary to hybridize with a template. The term “primer site” or “primer binding site” refers to the segment of the target DNA to which a primer hybridizes. The term “primer pair” means a set of primers including a 5′ “upstream primer” that hybridizes with the complement of the 5′ end of the DNA sequence to be amplified and a 3′ “downstream primer” that hybridizes with the 3′ end of the sequence to be amplified.

The term “target nucleic acid” refers to a nucleic acid (often derived from a biological sample), to which the probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid can refer to the specific subsequence of a larger nucleic acid to which the probe is directed or to the overall sequence (e.g., gene or mRNA) whose expression level it is desired to detect.

The term “complementary” means that one nucleic acid is identical to, or hybridizes selectively to, another nucleic acid molecule. Selectivity of hybridization exists when hybridization occurs that is more selective than total lack of specificity. Typically, selective hybridization will occur when there is at least about 55% identity over a stretch of at least 14-25 nucleotides, preferably at least 65%, more preferably at least 75%, and most preferably at least 90%. Preferably, one nucleic acid hybridizes specifically to the other nucleic acid. See M. Kanehisa, Nucleic Acids Res. 12:203 (1984).

The term “substantially complementary” means that a primer or probe need not be exactly complementary to its target sequence; instead, the primer or probe need be only sufficiently complementary to selectively hybridize to its respective strand at the desired annealing site. A non-complementary base or multiple bases can be included within the primer or probe, so long as the primer or probe retains sufficient complementarity with its polynucleotide binding site to form a stable duplex therewith.

A “perfectly matched probe” has a sequence perfectly complementary to a particular target sequence. The probe is typically perfectly complementary to a portion (subsequence) of a target sequence. The term “mismatch probe” refers to probes whose sequence is deliberately selected not to be perfectly complementary to a particular target sequence.

### Methods of Treatment

In certain embodiments, the present invention provides a method of treating a subject having a tumor that expresses EGFR and/or uPAR, even if at low levels, comprising: (a) administering intravenous fluids to the subject at a rate of about 0.1 to 1 ml/kg/hr for about 10 to 60 minutes, and (b) systemically administering a therapeutic composition at a dosage regime of 25 to 100 μg/kg, wherein the therapeutic composition is administered three times in a one-week period. In certain embodiments, the tumor is a hemangiosarcoma (HSA) or an angiosarcoma.

### Fluid-loading Procedure

In certain embodiments, the subject receives fluids prior to, concurrently with, and/or subsequent to administration of a drug. In one embodiment, the procedure for fluid loading before and/or during therapy in a subject is as follows:
Place peripheral intravenous catheter
Start intravenous fluids at 0.1 to 1 ml/kg/hr for 20 minutes prior and during drug administration
Connect the PhaSeal system
Administer drug SLOWLY over 20-25 minutes
Once infusion is complete, disconnect PhaSeal syringe
Let intravenous fluids continue to run for 2 minutes
Collect pharmacokinetics (if needed)
Pull intravenous catheter

### Methods of Prevention

In certain embodiments, the present invention provides a method of preventing hemangiosarcoma (HSA) in a dog predisposed to developing HSA or angiosarcoma in a human predisposed to developing angiosarcoma comprising: (a) administering intravenous fluids to the subject at a rate of about 0.1 to 1 ml/kg/hr for about 10 to 60 minutes, and (b) systemically administering a therapeutic composition at a dosage regime of 25 to 100 μg/kg, wherein the therapeutic composition is administered three times in a one-week period.

In certain embodiments, the present invention provides a method of preventing a hemangiosarcoma (HSA) in a dog that is positive for HSA by means of a blood test but negative by tumor imaging comprising: (a) administering intravenous fluids to the subject at a rate of about 0.1 to 1 ml/kg/hr for about 10 to 60 minutes, and (b) systemically administering a therapeutic composition at a dosage regime of 25 to 100 μg/kg, wherein the therapeutic composition is administered three times in a one-week period. In certain embodiments the blood test comprises: (a) providing a population of cells obtained from a blood sample from the dog; (b) determining (i) the level at which cells within the cell population concurrently express a plurality of cell markers, the plurality of cell markers comprising at least one primitive hematopoietic cell marker and at least one endothelial cell marker, and (ii) whether or not cells within the cell population express at least one leukemia cell marker or leukocyte-specific cell marker, wherein the at least one primitive hematopoietic cell marker is selected from the group consisting of CD117, CD34, and CD133; the at least one endothelial cell marker is selected from the group consisting of CD51/CD61, CD31, CD105, CD106 CD146 and von Willebrand Factor (vWF); and the at least one leukemia cell marker or leukocyte-specific cell marker is selected from the group consisting of CD18, CD3, CD5, CD21 and CD11b; and (c) comparing the level at which cells in the cell population concurrently express the plurality of cell markers with a control level of concurrent expression of the markers, wherein (1) an increase in the expression level of the plurality of cell markers relative to the control expression level, and (2) the absence of expression of CD18, CD3, CD5, CD21 and/or CD11b collectively are an indication of hemangiosarcoma (See, e.g., U.S. Pat. No. 7,910,315).

### Options for Detecting Markers

Expression of the various markers can be detected at the protein level by detecting the expressed proteins themselves, or at the transcript (i.e., mRNA) level by detecting transcript that encodes the corresponding proteins of interest. Conversely, proteins not expressed cannot be detected at the protein level or transcript level by the assays described below. Additional details regarding these various detection options follows.

### Antibodies for Use in Flow Cytometry and Other Immunological Methods

Antibodies to any of the markers described herein can be prepared according to routine methods that are known in the art (see, e.g., discussion below in the section on antibodies). Each antibody can also be obtained in purified form without a fluorochrome or biotin label, and labeled to any available fluorochrome in vitro using the AlexaFluor Zenon antibody labeling technology from Invitrogen/Molecular Probes, Eugene, Oreg. (emitting at 16 different wavelengths between 350 and 750 nm) or other equivalent technologies (e.g., Zymed and others). The resulting antibodies can be conjugated to any of a number of different labels, including for example, radioisotopes (e.g., ³H, ¹⁴C_{,} ³²P, ³⁵S, ¹²⁵I), fluorophores (e.g., pycoerythrin, fluorescein and rhodamine dyes and derivatives thereof), chromophores, chemiluminescent molecules, and enzyme substrates (e.g., the enzymes luciferase, alkaline phosphatase, beta-galactosidase and horse radish peroxidase).

Secondary detection systems employing an unlabeled antibody to bind to a cell marker and another labeled antibody to bind to the Fc region of the first antibody can be used in the immunoassays of the invention to increase the sensitivity of the assays.

In certain embodiments, the method described in U.S. Pat. No. 7,910,315 can be used.

### Therapeutic Compositions

A bispecific ligand-directed toxin (BLT), called EGFATFKDEL, consisting of human epidermal growth factor, a fragment of urokinase, and truncated pseudomonas exotoxin (PE38) was assembled in order to target human glioblastoma. Immunogenicity was reduced by mutating seven immunodominant B-cell epitopes on the PE38 molecule to create a new agent, EDFATFKDEL 7mut (WO 2012/027494 and Tsai et al., J. Neurooncol. 2011 103(2): 255-266, both of which are incorporated by reference herein).

Synthesis and assembly of hybrid genes encoding the single-chain EGFATFKDEL was accomplished using DNA-shuffling and DNA cloning techniques. The fully assembled fusion gene (from 5′ end to 3′ end) consisted of an NcoI restriction site, an ATG initiation codon, the genes for human EGF, the downstream 135-amino terminal fragment (ATF) from uPA linked by a 20 amino-acid segment of human muscle aldolase (HMA), the 7 amino-acid EASGGPE (SEQ ID NO:1) linker, the first 362 amino acids of the pseudomonas exotoxin (PE) molecule with KDEL (SEQ ID NO:2) replacing the REDLK (SEQ ID NO.3) at the C terminus, and a NotII restriction site at the 3′ end of the construct. The HMA segment was incorporated into the molecule as a flexible, non-immunogenic linker (Vallera D A, Todhunter D A, Kuroki D W, Shu Y, Sicheneder A, Chen H. A bispecific recombinant immunotoxin, DT2219, targeting human CD19 and CD22 receptors in a mouse xenograft model of B-cell leukemia/lymphoma. Clin Cancer Res. 2005; 11:3879-3888). The use of the ATF gene fragment was previously described by our laboratory (Rustamzadeh E, Hall W A, Todhunter D A, et al. Intracranial therapy of glioblastoma with the fusion protein DTAT in immunodeficient mice. Int J Cancer. 2006; 120:411-419; Vallera D A, Li C, Jin N, Panoskaltsis-Mortari A, Hall W A. Targeting urokinase-type plasminogen activator receptor on human glioblastoma tumors with diphtheria toxin fusion protein DTAT. J Nat Cancer Inst. 2002; 94:597-605). The resultant 1748 bp NcoI/NotII fragment gene was spliced into the pET28c bacteria expression vector under control of an isopropyl-b-D-thiogalactopyranoside inducible T7 promoter. DNA sequencing analysis (Biomedical Genomics Center, University of Minnesota) was used to verify that the gene was correct in sequence and had been cloned in frame. To create an EGFATFKDEL molecule with decreased immunogenicity, eight amino acids representing the seven major epitopes on PE38 were mutated using the Quick-Change Site-Directed Mutagenesis Kit (Stratagene. La Jolla Calif.). The following amino acids were altered: R490A, R513A, R467A, E548S, K590S, R432G, Q332S, R313A and confirmed by DNA sequencing. Genes for monospecific targeted toxins splicing PE38KDEL to human EGF (EGFKDEL) and mutated uPA fragment (ATFKDEL) were created using the same techniques. CD3CD3KDEL, a bispecific immunotoxin-targeting T-cell surface marker CD3, was made by replacing the DT₃₉₀ portion of the CD3CD3 (Bic3) molecule described previously with PE38KDEL (Vallera D A, Todhunter D, Kuroki D W, Shu Y, Sicheneder A, Panoskaltsis-Mortari A, Vallera V D, Chen H. Molecular modification of a recombinant, bivalent anti-human CD3 immunotoxin (Bic3) results in reduced in vivo toxicity in mice. Leuk Res. 2005; 29:331-341). 2219ARLKDEL, a BLT which combines VH and VL regions (sFv) for anti-CD22 and anti-CD19, was produced as described previously (Vallera D A, Chen H, Sicheneder A R, Panoskaltsis-Mortari A, Taras E P. Genetic alteration of a bispecific ligand-directed toxin targeting human CD19 and CD22 receptors resulting in improved efficacy against systemic B cell malignancy. Leuk Res. 2009; 33:1233-1242.).

In certain embodiments, the therapeutic composition comprises EGFATFKDEL mut7 described above. In certain embodiments, the therapeutic composition comprises EGFKDEL and ATFKDEL, wherein EGFKDEL and ATFKDEL are administered separately, simultaneously or sequentially. In certain embodiments, the dosage of the therapeutic composition is about 25-100 μg/kg.

### Pharmaceutical Compositions

The therapeutic compositions that are described herein, either in unconjugated form or conjugated form, can serve as the active ingredient in pharmaceutical compositions formulated for use in the various applications disclosed herein. These pharmaceutical compositions may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17th ed. 1985)).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, topically, intravenously, intraperitoneally, subcutaneously, intrathecally (for intracranial angiosarcoma) or intratumorally when the tumor is in the subcutaneous space. The formulations of compounds can be presented in unit-dose or multi-dose sealed containers, such as ampoules and vials. Solutions and suspensions can be prepared from sterile powders, granules, and tablets of the kind previously described.

The composition can be administered by means of an infusion pump, for example, of the type used for delivering chemotherapy to specific organs or tumors. Compositions of the inventions can be injected using a syringe or catheter directly into a tumor or at the site of a primary tumor prior to or after excision; or systemically following excision of the primary tumor. The compositions of the invention can be administered topically or locally as needed. For prolonged local administration, the enzymes may be administered in a controlled release implant injected at the site of a tumor. For topical treatment of a skin condition, the formulation may be administered to the skin in an ointment or gel.

The pharmaceutical compositions are particularly useful for parenteral administration, i.e., subcutaneously, intramuscularly or intravenously. The compositions for parenteral administration will commonly comprise a solution of a targeted compound (e.g., a ligand, ligand conjugate, antibody, or antibody conjugate) or a cocktail thereof dissolved in an acceptable carrier, preferably an aqueous carrier. In certain embodiments, the targeted compound is a ligand targeted toxin or an antibody targeted compound. A variety of aqueous carriers can be used, e.g., water, buffered water, phosphate buffered saline (PBS), 0.4% saline, 0.3% glycine, human albumin solution and the like. These solutions are sterile and generally free of particulate matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride and sodium lactate. The concentration of antibody in these formulations can vary widely, i.e., from less than about 0.005%, usually at least about 1% to as much as 15 or 20% by weight and will be selected primarily based on fluid volumes, viscosities, etc., in accordance with the particular mode of administration selected.

The dose administered to a subject should be sufficient to effect a beneficial response in the subject over time (e.g., to reduce tumor size or tumor load). Early detection may allow for prolonged remission/survival since the tumor would not yet be clinically evident and would be more amenable to control or elimination using the aforementioned treatments. The optimal dose level for any patient will depend on a variety of factors including the efficacy of the specific modulator employed, the age, body weight, physical activity, and diet of the patient, and on the severity of a particular disease. The size of the dose also will be determined by the existence, nature, and extent of any adverse side-effects that accompany the administration of a particular compound or vector in a particular subject. In certain embodiments, the therapeutic composition is administered by means of a slow IV push. In certain embodiments, the composition is administered by means of a syringe or infusion pump and delivered over a period, which can be 30 minutes or longer. In certain embodiments, the administration is for about three hours.

### Treatment Methods

Once a subject has been diagnosed using the methods provided herein as having an elevated risk of tumor, various treatment options can be implemented. One option is to conduct surgery to try to excise the tumor (if a tumor mass is grossly detectable) using standard surgical procedures in the art. Another option is to begin chemotherapy to try to eradicate the tumor. Of course combined treatment regimens using both surgery and chemotherapy can be implemented.

In certain embodiments, the methods disclosed herein can be used “prophylactically” in that they can be used to detect “tumor cells” before the tumor is clinically detectable using existing state-of-the-art techniques. This means that treatment (e.g., administration of ligand targeted toxins, such as described herein, or antibodies) need not be administered blindly simply to ward off the disease. Rather treatments can be tailored to the subject's particular needs when the disease is still at a microscopic stage, thereby increasing the ability to prevent the tumor from progressing to clinically evident disease.

In therapeutic applications, compositions (e.g., the pharmaceutical compositions provided herein) are administered to a subject that already has been diagnosed as having a hemangiosarcoma or an angiosarcoma (e.g., using the methods provided herein). The composition is administered in an amount sufficient to cure or at least partially arrest the disease and its complications (e.g., to reduce the tumor size or arrest its spread). An amount adequate to accomplish this is defined as a “therapeutically effective dose.” Amounts effective for this use will depend upon the severity of the disease, the extent to which the tumor has metastasized, the age and weight of the subject, and other factors known to those of skill in the art, but generally range from about 1 to about 200 mg of antibody per dose, with dosages of from 5 to 70 mg per patient being more commonly used. Dosing schedules will vary with the disease state and status of the patient, and will typically range from a single bolus dosage or continuous infusion to multiple administrations per day (e.g., every 4-6 hours), or as indicated by the treating medical professional and the patient's condition.

It must be kept in mind that the materials of this invention may generally be employed in serious disease states, that is, life-threatening or potentially life-threatening situations. In such cases, in view of the minimization of extraneous substances and the lower probability of “foreign substance” rejections which are achieved using certain antibodies described herein (e.g., chimeric or humanized antibodies), it is possible, and may be felt desirable by the treating clinician, to administer substantial excesses of these therapeutic compositions.

In certain embodiments, the therapy is administered in a “cycle.” In certain embodiments a “cycle” of drug therapy equals three treatments on every other day schedule, such as Monday, Wednesday and Friday. In certain embodiments, the biologically active dose is about 20-100 50 μg/kg. In certain embodiments, the dose is about 50 μg/kg. In certain embodiments, more than one “cycle” is administered, e.g., two, three or four cycles. In certain embodiments, the cycles are administered during consecutive weeks. In certain embodiments, the cycles are administered with a week or more (e.g., a month or longer) between each cycle. In certain embodiments, repeated dosing improves efficacy without enhancing toxicity.

In certain embodiments, the therapy is administered daily. In certain embodiments, the administration is for a duration of at least six months.

In certain embodiments, the administration is by systemic administration, and specifically by means of intravenous administration.

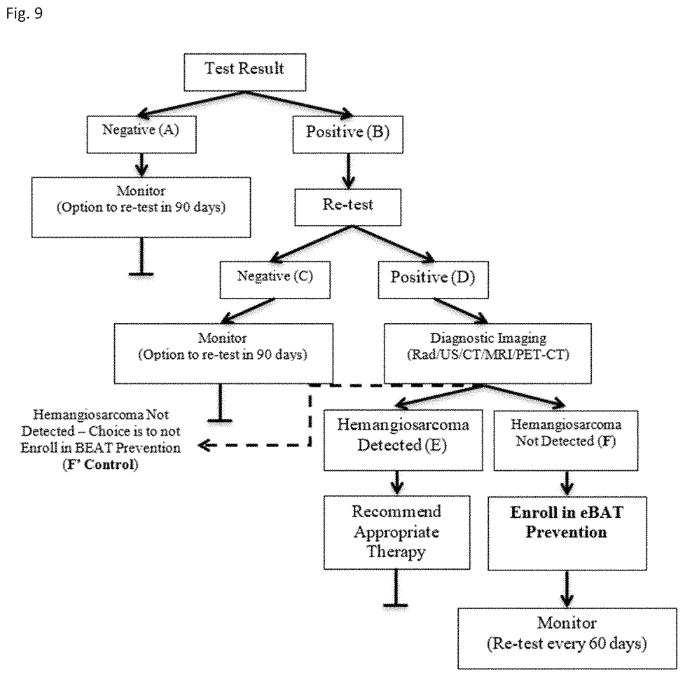

In certain embodiments, the flow-steps shown in FIG. 9 are followed to treat dogs at risk (could be any dog at risk based on genetic background) as well as to treat dogs or humans at risk because they have tested positive for circulating tumor cells.

The following example is intended to illustrate but not limit the invention.

### EXAMPLE

### Safe and Effective Sarcoma Therapy through Bispecific Targeting of EGFR and uPAR Abstract

Sarcomas differ from carcinomas in their mesenchymal origin. Therapeutic advancements have come slowly so alternative drugs and models are urgently needed. These studies report a new drug for sarcomas that simultaneously targets both tumor and the tumor neovasculature. eBAT is a bispecific angiotoxin consisting of truncated, deimmunized Pseudomonas exotoxin fused to epidermal growth factor (EGF) and the amino terminal fragment (ATF) of urokinase. Here, we study the drug in an in vivo “ontarget” companion dog trial since eBAT effectively kills canine hemangiosarcoma (HSA) and human sarcoma cells in vitro. We reasoned the model has value due to the common occurrence of spontaneous sarcomas in dogs and a limited lifespan allowing for rapid accrual and data collection. Splenectomized dogs with minimal residual disease were given one cycle of eBAT followed by adjuvant doxorubicin in an adaptive dose-finding, phase I-II study of 23 dogs with spontaneous, stage I-II, splenic HSA. eBAT improved 6-month survival from <40% in a historical comparison population to ˜˜70% in dogs treated at a biologically active dose (50 μg/kg). Five dogs were cured. Surprisingly, eBAT abated expected toxicity associated with EGFR-targeting, a finding supported by mouse studies. The literature validates uPAR and EGFR as targets for human sarcomas, so thorough evaluation is crucial for validation of the dog model. Thus, we confirmed the validity of these markers for human sarcoma targeting in the study of 212 human and 97 canine sarcoma samples. Our results support further translation of eBAT for human sarcoma patients and perhaps other EGFR-expressing malignancies.

### Introduction

Unlike carcinomas derived from epithelial tissues, sarcomas comprise a heterogeneous group of malignancies of mesenchymal origin. There are 15,000 new sarcoma cases per year in the United States, consisting of 12,000 cases of soft tissue sarcoma and 3,000 cases of bone sarcomas. The 5-year overall survival rate is approximately 50-80% for sarcomas. Development of new targeted therapies for therapy-resistant sarcoma has suffered from the lack of widely-expressed mutations or overexpressed proteins that can be targeted therapeutically without risk of severe adverse events.

eBAT, a bispecific EGF-urokinase angiotoxin, was developed as a targeted, second generation bispecific biologic drug consisting of human EGF (targeting EGFR), human amino terminal transferase (ATF is the high affinity binding moiety of human urokinase, targeting uPAR), and genetically modified Pseudomonas exotoxin, mutated to reduce immunogenicity and facilitate ER retention. This drug was highly efficacious in treatment of established glioma in rodent xenograft models (Tsai A K, Oh S, Chen H, et al. A novel bispecific ligand-directed toxin designed to simultaneously target EGFR on human glioblastoma cells and uPAR on tumor neovasculature. J Neurooncol 2011; 103(2):255-66). Xenograft models are informative, but targeting human cells in “non-target” immunosuppressed mice (that do not bind human EGF and ATF) does not yield the same clinical investigative information as studies in a large animal “ontarget” models where the drug cross-reacts with native EGFR and uPAR. Thus, we chose to undertake an “ontarget” clinical trial in companion dogs with hemangiosarcoma (HSA).

Canine HSA is a common, aggressive, incurable spontaneous sarcoma with a pathogenesis similar to human idiopathic angiosarcoma. Microscopic metastases are commonly present at diagnosis, and treatment is often unrewarding in both diseases. Similarly to humans with angiosarcoma who have an expected median survival of approximately 16 months, dogs with HSA have a short median survival when treated with the standard-of-care of surgery and adjuvant chemotherapy. Morbidity and mortality are usually caused by metastatic spread and/or acute internal hemorrhage secondary to tumor rupture. It was hypothesized that HSA is a vascular cancer and eBAT simultaneously targeting the tumor and its vasculature rendered it an excellent therapy choice.

Expression of EGFR and PLAUR/uPAR was previously characterized in human sarcomas using conventional PCR-based assays, gene expression microarrays, and immunohistochemistry (Albritton K H, Randall R L. Prospects for targeted therapy of synovial sarcoma. J Pediatr Hematol Oncol 2005; 27(4):219-22; Yang J L, Hannan M T, Russell P J, et al. Expression of HER1/EGFR protein in human soft tissue sarcomas. Eur J Surg Oncol 2006; 32(4):466-8; Tschoep K, Kohlmann A, Schlemmer M, et al. Gene expression profiling in sarcomas. Crit Rev Oncol Hematol 2007; 63(2):111-24; Benassi MS, Ponticelli F, Azzoni E, et al. Altered expression of urokinase-type plasminogen activator and plasminogen activator inhibitor in high-risk soft tissue sarcomas. Histol Histopathol 2007; 22(9):1017-24). In this work, it was confirmed such expression in a variety of human sarcomas and report on EGFR and uPAR expression on canine HSA.

It is shown that canine HSA tumor-initiating cells express EGFR and uPAR, and that these cells are highly sensitive to eBAT (Tsai A K, Oh S, Chen H, et al. A novel bispecific ligand-directed toxin designed to simultaneously target EGFR on human glioblastoma cells and uPAR on tumor neovasculature. J Neurooncol 2011; 103(2):255-66; Mazar A P, Ahn R W, O'Halloran T V. Development of novel therapeutics targeting the urokinase plasminogen activator receptor (uPAR) and their translation toward the clinic. Curr Pharm Des 2011; 17(19):1970-8; Schappa J T, Frantz A M, Gorden B H, et al. Hemangiosarcoma and its cancer stem cell subpopulation are effectively killed by a toxin targeted through epidermal growth factor and urokinase receptors. Int J Cancer 2013; 133(8):1936-44; Waldron N N, Oh S, Vallera D A. Bispecific targeting of EGFR and uPAR in a mouse model of head and neck squamous cell carcinoma. Oral Oncol 2012; 48(12):1202-7). Here, a large “ontarget” animal study was used that closely parallels what could be human clinical trial to show feasibility, safety, and efficacy of eBAT to treat sarcomas in a clinically translatable setting using spontaneous canine HSA as model, in both naive disease and minimal residual disease settings. The impact of bispecific targeting on the toxicity risks associated with targeting of EGFR is reported herein. The results show that eBAT is safe and effective at biologically active doses despite EGFR targeting, supporting further translation for patients with sarcomas and other EGFR-expressing malignancies. Furthermore, our findings support our belief that bispecificity reduces overall toxicity risks associated with EGFR targeting.

### Materials and Methods

### Assessment of EGFR and PLAUR/uPAR Expression in Human and Canine Tumors

EGFR and PLAUR mRNA expression was evaluated from data for 212 human sarcomas obtained through the TCGA (The Cancer Genome Atlas) Research Network (http://cancergenome.nih.gov/). The federal project was begun in 2005 to catalog genetic mutations responsible for cancer using genome sequencing and bioinformatics. The goal of analyzing EGFR and PLAUR expression was in line with its goal of helping researchers generate statistically and biologically significant conclusions from its extensive genomic database. To perform a similar analysis in dogs, a Genome-wide Association Study was used utilizing next-generation RNA sequencing (RNAseq) datasets for canine hemangiosarcoma and canine lymphoma reported previously (Gorden B H, Kim J H, Sarver A L, et al. Identification of three molecular and functional subtypes in canine hemangiosarcoma through gene expression profiling and progenitor cell characterization. Am J Pathol 2014; 184(4):985-95; Tonomura N, Elvers I, Thomas R, et al. Genome-wide Association Study Identifies Shared Risk Loci Common to Two Malignancies in Golden Retrievers. PLoS Genet 2015; 11(2):e1004922). RNAseq for 31 canine osteosarcoma samples was performed as described (Gorden B H, Kim J H, Sarver A L, et al. Identification of three molecular and functional subtypes in canine hemangiosarcoma through gene expression profiling and progenitor cell characterization. Am J Pathol 2014; 184(4):985-95, Temiz N A, Moriarity B S, Wolf N K, et al. RNA sequencing of Sleeping Beauty transposon-induced tumors detects transposon-RNA fusions in forward genetic cancer screens. Genome Res 2016; 26(1):119-29; Sarver A E, Sarver A L, Thayanithy V, et al. Identification, by systematic RNA sequencing, of novel candidate biomarkers and therapeutic targets in human soft tissue tumors. Lab Invest 2015; 95(9):1077-88). EGFR and uPAR protein expression were evaluated in a human synovial sarcoma tissue microarray (TMA) (Charbonneau B, Vogel R I, Manivel J C, et al. Expression of FGFR3 and FGFR4 and clinical risk factors associated with progression-free survival in synovial sarcoma. Hum Pathol 2013; 44(9):1918-26); the same methods were used to build a study-specific TMA that included tumors from 15 dogs as well as normal canine spleen, liver and kidney and spleens with nodular lymphoid hyperplasia and associated hematomas as controls. A total of 97 canine sarcoma samples were analyzed (51 HSAs from the TCGA, 31 osteosarcomas from the TCGA, and 15 HSAs from dogs enrolled in our clinical study). IHC methods are provided below.

### Cell Lines

The Emma canine HSA cell line was cultured in hemangiosarcoma medium as described (Schappa J T, Frantz A M, Gorden B H, et al. Hemangiosarcoma and its cancer stem cell subpopulation are effectively killed by a toxin targeted through epidermal growth factor and urokinase receptors. Int J Cancer 2013; 133(8):1936-44; Tamburini B A, Trapp S, Phang T L, et al. Gene expression profiles of sporadic canine hemangiosarcoma are uniquely associated with breed. PLoS One 2009; 4(5):e5549). The human RH-30 alveolar rhabdomyosarcoma cell line (COG), RD (ATCC) embryonal rhabdomyosarcoma cell line, TC-71 Ewing sarcoma cell line (COG), and U2OS osteosarcoma cell line (ATCC) were grown in Dulbecco's modified Eagle's Medium (DMEM) as described (Li L, Sarver A L, Alamgir S, et al. Downregulation of microRNAs miR-1, -206 and -29 stabilizes PAX3 and CCND2 expression in rhabdomyosarcoma. Lab Invest 2012; 92(4):571-83; Huang X, Park H, Greene J, et al. IGF1R- and ROR1-Specific CAR T Cells as a Potential Therapy for High Risk Sarcomas. PLoS One 2015; 10(7):e0133152; Scott M C, Sarver A L, Tomiyasu H, et al. Aberrant Retinoblastoma (RB)-E2F Transcriptional Regulation Defines Molecular Phenotypes of Osteosarcoma. J Biol Chem 2015; 290(47):28070-83). All cell lines were authenticated using short tandem repeat profiles (DNA Diagnostics Center, Inc., Fairfield, Ohio).

### eBAT Production

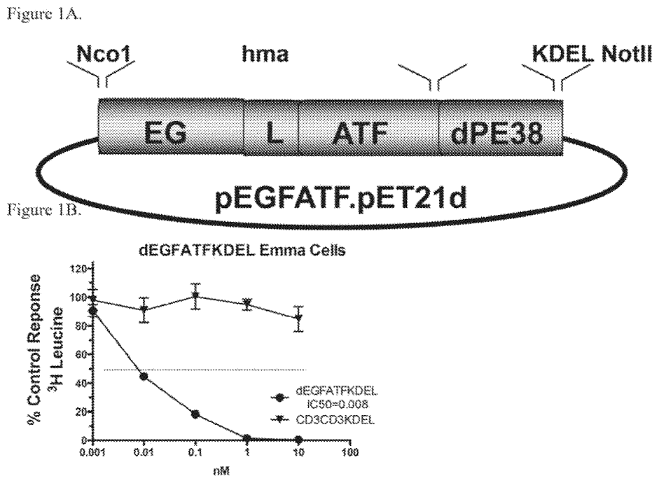

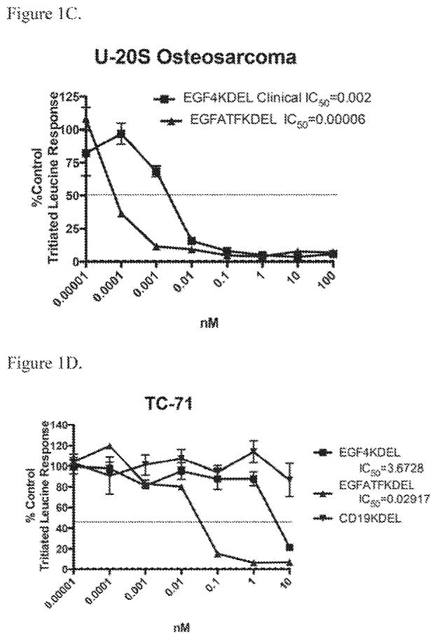

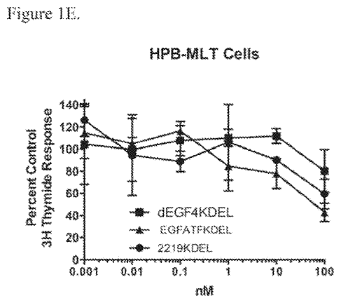

eBAT was produced at the University of Minnesota cGMP Molecular and Cellular Therapeutics (MCT) Facility as described (Tsai A K, Oh S, Chen H, et al. A novel bispecific ligand-directed toxin designed to simultaneously target EGFR on human glioblastoma cells and uPAR on tumor neovasculature. J Neurooncol 2011; 103(2):255-66). The construction of eBAT is illustrated in FIG. 1A. Release assays were done by Pace Analytical Life Sciences, LLC (Minneapolis, Minn.) and/or at the MCT. Release criteria were established regarding drug purity (>95%), endotoxin (<50 Eu/mg), stability, selectivity, potency (IC50<1.0 nM), sterility, and concentration. The drug was vialed and re-tested to meet critical FDA specifications.

### Laboratory Assays

Protein synthesis assays measuring [³H]leucine incorporation were used to determine the effect of eBAT on cell lines. Proliferation assays were performed measuring the incorporation of [³H]thymidine. Briefly, cells are plated in 96-well flat-bottomed plates and allowed to adhere overnight. The targeted toxins were added in triplicate at 10-fold serial dilutions and incubated for 48 hours. Wells are then pulsed with either [³H]leucine (protein synthesis assay) or [³H]thymidine (proliferation assay) with 1 μCi per well and allowed to incubate for another 24 hours. Plates are then frozen to detach the cells, harvested onto glass fiber filters, washed, dried, and counted using standard scintillation methods. [³H]leucine assays were performed using Leucine-free medium. Data are reported as the percentage of control counts.

To evaluate safety, C57BL/6 mice were administered eBAT by the intraperitoneal route twice, two days apart on days 1 and day 3, and then were observed for adverse events for 3 weeks.

### Canine Clinical Study

Safety and efficacy of adjuvant eBAT were assessed using a Bayesian adaptive Phase I-II trial design with pre-defined criteria of acceptable toxicity (no dose-limiting adverse events) and efficacy (>50% survival at 6 months) to guide dose finding (Koopmeiners J S, Modiano J. A Bayesian adaptive Phase I-II clinical trial for evaluating efficacy and toxicity with delayed outcomes. Clin Trials 2014; 11(1):38-48). eBAT was administered to dogs with spontaneous HSA after splenectomy and before the first of five cycles of doxorubicin chemotherapy. Eligibility was restricted to dogs with stage-1 or stage-2 splenic HSA with no evidence of gross metastatic disease. Adverse events (AEs) were graded according to VCOG-CTCAE criteria (Vail D M. Veterinary Co-operative Oncology Group-Common Terminology Criteria for Adverse Events (VCOG-CTCAE) following chemotherapy or biological antineoplastic therapy in dogs and cats v1.0. Vet Comp Oncol 2004; 2(4):195-213). Survival time was measured from the date of diagnosis to the time of death and was censored at the time of last contact for dogs surviving at the time of analysis.

The clinical study, called SRCB ST-1 (sarcoma bispecific toxin trial-1), was conducted with approval of the University of Minnesota (UMN) Institutional Animal Care and Use Committee (IACUC Protocols 1110A06186 and 1507-32804A). Study design and implementation conformed to Consolidated Standards of Reporting Trails (CONSORT) guidelines as they apply to studies in companion animals (Hinchcliff K W, DiBartola S P. Quality matters: publishing in the era of CONSORT, REFLECT, and EBM. J Vet Intern Med 2010; 24(1):8-9.). eBAT pharmacokinetics and neutralizing antibody assays were performed for all dogs. Detailed descriptions of the comparison group, eligibility criteria and protocols for the SRCBST-1 study, pharmacokinetics and neutralizing antibody assays are provided below.

### Detailed Methods for Immunohistochemistry

Immunohistochemical staining for EGFR and uPAR in human and canine tissues was optimized for Aperio quantification as described (Charbonneau B, Vogel R I, Manivel J C, Rizzardi A, Schmechel S C, Ognjanovic S, et al. Expression of FGFR3 and FGFR4 and clinical risk factors associated with progression-free survival in synovial sarcoma. Hum Pathol. 2013; 44:1918-26; Rizzardi A E, Johnson A T, Vogel R I, Pambuccian S E, Henriksen J, Skubitz A P, et al. Quantitative comparison of immunohistochemical staining measured by digital image analysis versus pathologist visual scoring. Diagnostic pathology. 2012; 7:42). Vimentin staining (Zymed cat#18-0052 clone V9 mouse monoclonal (ThermoFisher) was used as a control for viable tissue; regions of negative staining were excluded from image analysis. CD31 staining (CD31 Dako, cat#M0823, Clone JC70A, http://www.dako.com/us/download.pdf?objectid=102467002) was used to localize the regions of tumor used for analysis and quantification. Antibody was used at a 1:100 for dilution. Heat retrieval was done prior to staining in citrate buffer (pH 6.) for 30 minutes with 20 minutes cool down prior to staining. The antibodies used to stain EGFR and uPAR, respectively, were anti-EGFR precursor antibody produced in rabbit (Sigma Prestige cat# HPA018530, http://www.sigmaaldrich.com/catalog/product/sigma/hpa018530?lang=en&region=US), and clone R4 mouse anti-uPAR monoclonal antibody (Dako cat# M7294, http://www.dako.com/us/download.pdf?obj ectid=114115003). Methods for immunohistochemistry were the same for all antibodies.

### Detailed Description of the Canine Clinical Study

Owners of each dog gave written informed consent to treat prior to study entry. All dogs were treated at the University of Minnesota Veterinary Medical Center (VMC); the study was managed by the Clinical Investigation Center (CIC) of the College of Veterinary Medicine, University of Minnesota in compliance with principles of Good Clinical Practice.

Inclusion criteria included histopathologically-confirmed diagnosis of stage-I (no evidence of tumor rupture) or stage-II (evidence of tumor rupture) HSA confined to the spleen; no evidence of regional or distant metastatic disease based on thoracic radiography and abdominal ultrasonography that was grossly confirmed at the time of surgery; no concurrent treatment with herbal treatments or supplements; performance score of 0 or 1 according to the Karnofsky's modified performance scale (Karnofsky D A, Abelmann W H, Craver L F, Burchenal J H. The use of the nitrogen mustards in the palliative treatment of carcinoma. With particular reference to bronchogenic carcinoma. Cancer. 1948; 1:634-56); adequate organ function; no serious comorbidities, such as renal or hepatic failure, congestive heart failure, or clinical coagulopathy.

Dogs were required to have a splenectomy prior to study entry. Each dog received a baseline complete history, physical examination, and pre-dose laboratory assessment that included a complete blood count (CBC), serum biochemical profile, coagulation parameters (PT/PTT) and urinalysis. Thoracic radiography and abdominal ultrasonography were performed prior to enrollment to rule out gross metastatic disease. eBAT was administered in a single cycle of three intravenous treatments at days 1, 3, and 5 at escalating doses of 25 μg/kg/day (dose 1), 50 μg/kg/day (dose 2), or 100 μg/kg/day (dose 3). The protocol was modified starting with the fifth dog to include pre-loading with intravenous fluids at a rate of about 0.1 to 1 ml/kg/hr for 10 to 60 minutes. The drug was administered as a slow infusion over 5-20 minutes depending on volume and size of the dog.

In total, the investigators were in contact with 181 families via email or telephone to assess their dog's eligibility to participate in SRCBST-1. Of these, 79 dogs had surgery to remove a grossly abnormal spleen between Nov. 28, 2012 and May 6, 2015 (51 at the VMC and 28 at another hospital prior to referral) and 23 dogs were enrolled in the study. One of these 23 dogs was euthanized at study Day 18 due to metastatic dissemination to the liver with rupture and hemoabdomen. This dog did not receive doxorubicin chemotherapy, but was included in all the analyses.

Disease reassessment included physical examination, blood and urine evaluation, thoracic radiography and abdominal ultrasonography prior to doxorubicin treatments #3 and #5. No medications were prescribed or administered concurrently, unless needed to manage toxicity or other, unrelated medical conditions. Adverse events related to the study drug or to doxorubicin chemotherapy were treated with supportive care, as needed. Gastrointestinal toxicities were managed with famotidine, omeprazole, metronidazole, metoclopramide, ondansetron, and/or maropitant. Antibiotic therapy was allowed for prophylaxis in the event of severe neutropenia (counts <1,000/μl) or febrile neutropenia. Non-steroidal anti-inflammatory drugs or other analgesics (tramadol, gabapentin) were allowable for pain control as needed.

Baseline characteristics for all dogs and by dose are summarized in Table 1A. The study protocol is summarized in Table 1B. The most common laboratory abnormalities at the time of screening included mild regenerative anemia (11 dogs), thrombocytosis (19 dogs), mild to moderate ALP elevation (six dogs), isosthenuria (four dogs), and slight ALT and AST elevation or slight hypoalbuminemia or proteinuria (one dog each). Slides were available for review by one of the study pathologists for 15 dogs. Most of the cases had mixed morphology, with areas showing capillary, cavernous, or solid organization. Mitotic indices also were comparable for each of these cases.

TABLE 1A
Baseline characteristics for all dogs and by dose summarized by N (%) or mean (SD)

| Variable | Level/Unit | All Dogs | | Dose 1 | | Dose 2 | | Dose 3 | |
|---|---|---|---|---|---|---|---|---|---|
| Breed | German Shepherd Mix | 2 | (8.6%) | 1 | (33.3%) | 1 | (5.9%) | 0 | (0%) |
| | Labrador Retriever | 5 | (21.7%) | 1 | (33.3%) | 4 | (23.5%) | 0 | (0%) |
| | English Setter | 1 | (4.3%) | 1 | (33.3%) | 0 | (0%) | 0 | (0%) |
| | Brittany Spaniel | 1 | (4.3%) | 0 | (0%) | 1 | (5.9%) | 0 | (0%) |
| | Airedale Terrier | 1 | (4.3%) | 0 | (0%) | 1 | (5.9%) | 0 | (0%) |
| | Bichon Frise | 1 | (4.3%) | 0 | (0%) | 1 | (5.9%) | 0 | (0%) |
| | Newfoundland | 1 | (4.3%) | 0 | (0%) | 0 | (0%) | 1 | (33.3%) |
| | Viszla | 1 | (4.3%) | 0 | (0%) | 0 | (0%) | 1 | (33.3%) |
| | Goldendoodle | 1 | (4.3%) | 0 | (0%) | 0 | (0%) | 1 | (33.3%) |
| | English Springer Spaniel | 2 | (8.7%) | 0 | (0%) | 2 | (11.8%) | 0 | (0%) |
| | Cairn Terrier | 1 | (4.3%) | 0 | (0%) | 1 | (5.9%) | 0 | (0%) |
| | Papillon | 1 | (4.3%) | 0 | (0%) | 1 | (5.9%) | 0 | (0%) |
| | Mixed breed | 1 | (4.3%) | 0 | (0%) | 1 | (5.9%) | 0 | (0%) |
| | Dachshund | 1 | (4.3%) | 0 | (0%) | 1 | (5.9%) | 0 | (0%) |
| | Golden Retriever | 1 | (4.3%) | 0 | (0%) | 1 | (5.9%) | 0 | (0%) |
| | Rat Terrier | 1 | (4.3%) | 0 | (0%) | 1 | (5.9%) | 0 | (0%) |
| | German Shepherd | 1 | (4.3%) | 0 | (0%) | 1 | (5.9%) | 0 | (0%) |
| Age | Years | 9.4 | (1.7) | 9.2 | (1.6) | 9.5 | (1.8) | 8.7 | (1.8) |
| Sex | M | 11 | (47.8) | 1 | (33.3) | 9 | (52.9) | 1 | (33.3) |
| | F | 12 | (52.2) | 2 | (66.7) | 8 | (47.1) | 2 | (66.7) |
| BCS | | 5.7 | (1) | 5.7 | (0.6) | 5.5 | (1.1) | 6.3 | (0.6) |
| Weight | kg | 24.6 | (11.7) | 30.1 | (4.6) | 22.1 | (12.1) | 33.3 | (9.1) |
| Hemoabdomen | Y | 20 | (87) | 2 | (66.7) | 15 | (88.2) | 3 | (100) |
| | N | 3 | (13) | 1 | (33.3) | 2 | (11.8) | 0 | (0) |
| Stage | 1 | 2 | (8.7) | 0 | (0) | 2 | (11.8) | 0 | (0) |
| | 2 | 20 | (87) | 2 | (66.7) | 15 | (88.2) | 3 | (100) |
| | 3 | 1 | (4.3) | 1 | (33.3) | 0 | (0) | 0 | (0) |
| Time from surgery | Days | 22.8 | (10.6) | 15 | (5.2) | 25.1 | (11.4) | 18 | (2) |
| to treatment | | | | | | | | | |
| Time to Initiation | Days | 43.7 | (11.3) | 35.3 | (5.5) | 46.2 | (12.2) | 38.7 | (3.1) |
| of Chemotherapy | | | | | | | | | |

A historical comparison group (Comparison group) consisted of 28 dogs with stage-I (8 dogs) or stage-II (20 dogs) HSA treated with SOC alone (surgery followed by adjuvant chemotherapy) at the MC between 2005 and 2014. Chemotherapy treatment in this group consisted of metronomic piroxicam and cyclophosphamide in 2 dogs, doxorubicin chemotherapy in 8 dogs, and doxorubicin chemotherapy combined with metronomic cyclophosphamide in 20 dogs. Of these 20 dogs, one was switched to a CCNU/dacarbazine regimen due to progressive disease, and in another dog metronomic chlorambucil was used to replace cyclophosphamide following the development of sterile hemorrhagic cystitis. Survival times for all dogs were calculated from the date of diagnosis to the date of death.

### eBAT Pharmacokinetics and Neutralizing Antibody Assays

Serum samples were collected before starting treatment (time 0) and 5, 15, 30, 45, and 60 minutes after the end of the infusion on days 1 and 5 or 6 to measure drug pharmacokinetics (PK). Single serum samples were collected on days 8 and 21 to assess the presence of neutralizing antibodies (NAs). A bioassay with human RD cells was used to measure eBAT PK, as reported. Cells were incubated overnight prior to addition of the clinical batch eBAT as reported (Waldron N N, Oh S, Vallera D A. Bispecific targeting of EGFR and uPAR in a mouse model of head and neck squamous cell carcinoma. Oral Oncol 2012; 48(12):1202-7). Proliferation was measured after 72 hours using a standard thymidine uptake assay. The presence of eBAT in serum was extrapolated from the standard curve as reported (Stish B J, Oh S, Chen H, Dudek A Z, Kratzke R A, Vallera D A. Design and modification of EGF4KDEL 7Mut, a novel bispecific ligand-directed toxin, with decreased immunogenicity and potent anti-mesothelioma activity. British Journal of Cancer (2009) 101, 1114-1123. doi:10.1038/sj.bjc.6605297). The presence of NAs was inferred from the capacity of serum samples to block cell death caused by reference eBAT (Oh S, Stish B J, Sachdev D, Chen H, Dudek A Z, Vallera D A. A novel “reduced immunogenicity” bispecific targeted toxin simultaneously recognizing human EGF and IL-4 receptors in a mouse model of metastatic breast carcinoma Clin Cancer Res. 2009 Oct. 1; 15(19): 6137-6147. doi: 10.1158/1078-0432.CCR-09-0696).

### Statistical Analysis

Univariate associations between time-to-death and gene expression, patient characteristics and tumor characteristics for the TCGA samples were assessed by cox proportional hazard regression and summarized by Kaplan-Meier curves. Associations between time-to-death and expression of EGFR or uPAR were assessed by multivariate cox regression analysis, and adjusted for each other and for patient and tumor characteristics. Associations between EGFR and uPAR expression in human and canine tumor samples were evaluated using Pearson's correlation coefficient.

Dogs and disease characteristics were summarized using descriptive statistics. The biologically active dose was identified as specified by the design (Koopmeiners J S, Modiano J. A Bayesian adaptive Phase I-II clinical trial for evaluating efficacy and toxicity with delayed outcomes. Clin Trials 2014; 11(1):38-48). Model-based estimates of the probability of AEs and 6-month survival were obtained from the parametric model used to guide dose finding. The probability of AEs was estimated using a logistic regression model with a linear term for dose; the probability of 6-month survival was modeled using a logistic regression model with linear and quadratic terms for dose. The probability of AEs for each dose was estimated by the sample proportion with exact confidence intervals. Kaplan-Meier curves for overall survival were fit for the entire study population and only for dogs treated at the biologically active dose to obtain a non-parametric estimate of 6-month survival and median time-to-death. Associations between AEs and baseline covariates of age, weight and body condition score (BCS) were assessed using the t-test. All analyses were performed using R version 3.0.1 (R Core Team. R: A Language and Environment for Statistical Computing. In: R: A Language and Environment for Statistical Computing. R Foundation for Statistical Computing; 2013).

### Results

### eBAT Inhibits Activity of Canine and Human Sarcoma Cells

To assess activity, eBAT was added to EGFR+uPAR+ canine EMMA cells and leucine incorporation was measured as an indication of protein synthesis activity (FIG. 1B). The activity of EMMA cells was inhibited in a dose-dependent manner and was specific since a control anti-human CD3 targeted toxin, CD3CD3KDEL, recognizing the epsilon chain of the T cell receptor did not have activity. In FIG. 1C, U-20S human osteosarcoma cells were inhibited by eBAT in a thymidine incorporation assay that measures proliferation, rather than protein synthesis. Interestingly, a bispecific targeted toxin EGF4KDEL (Stish B J, Oh S, Chen H, Dudek A Z, Kratzke R A, Vallera D A. Design and modification of EGF4KDEL 7Mut, a novel bispecific ligand-directed toxin, with decreased immunogenicity and potent anti-mesothelioma activity. British Journal of Cancer (2009) 101, 1114-1123, doi:10.1038/sj.bjc.6605297 www.bjcancer.com; Oh S, Stish B J, Sachdev D, Chen H, Dudek A Z, Vallera D A. A novel “reduced immunogenicity” bispecific targeted toxin simultaneously recognizing human EGF and IL-4 receptors in a mouse model of metastatic breast carcinoma Clin Cancer Res. 2009 Oct. 1; 15(19): 6137-6147. doi: 10.1158/1078-0432.CCR-09-0696) that simultaneously targets EGFR and the human IL-4 receptor did not inhibit as well. Similar findings were observed when TC71 Ewing's Sarcoma cells were targeted in FIG. 1D. Again, the CD3CD3KDEL control did not inhibit. The IC50 (concentration inhibiting 50% drug activity for all of these cell lines was in the subnanomolar range (0.06 pM-0.08 nM). eBAT was also tested against negative control EGFR-uPAR-human HPB-MLT T-cells showing no significant cytotoxicity as expected (FIG. 1E). Together, these findings indicate that eBAT is extremely potent and inhibits both protein synthesis and DNA synthesis in a highly specific manner in vitro.

### Human Sarcomas Express Epidermal Growth Factor Receptor and Urokinase Receptor

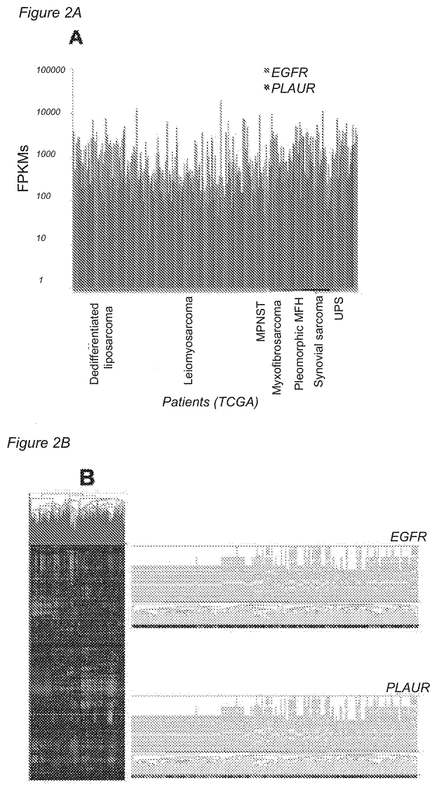

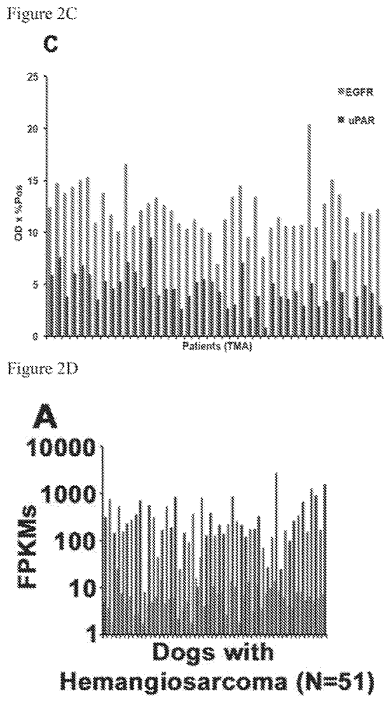

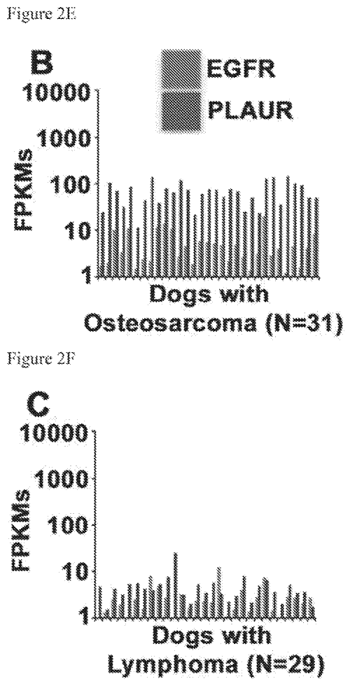

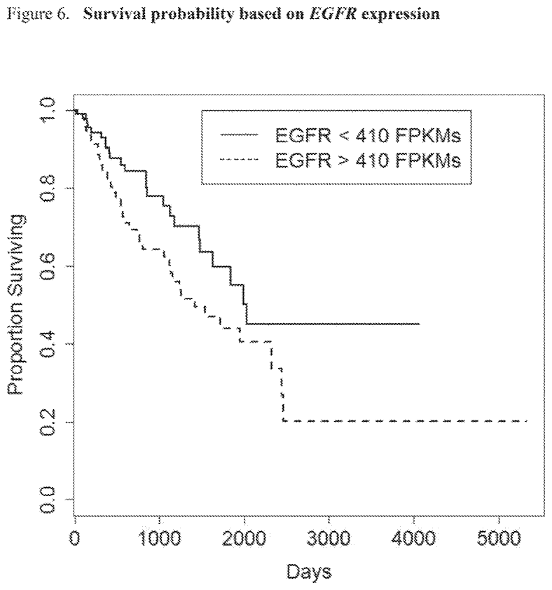

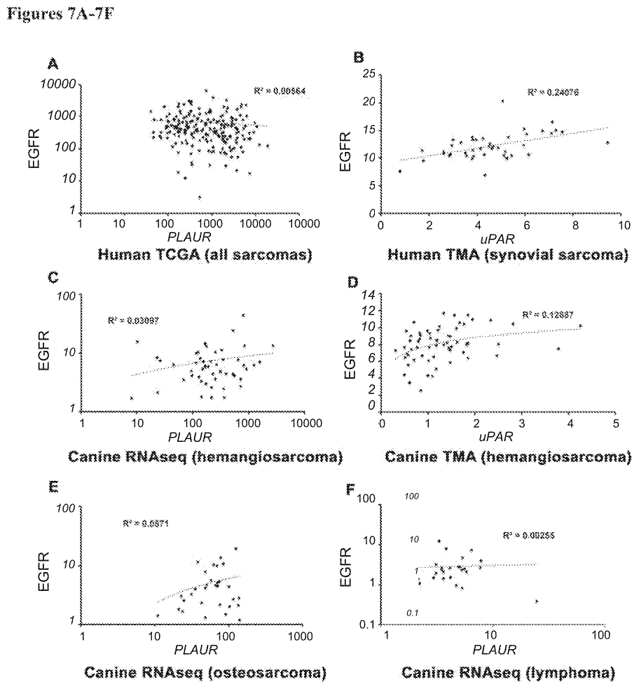

The most current bioinformatics TCGA database was used to explore the expression on of EGFR and PLAUR on 212 sarcomas human sarcomas (FIG. 2). FIG. 2A shows that EGFR and PLAUR gene expression were detectable in 100% of samples regardless of sarcoma type with a variation in intensity. FIG. 6 shows Kaplan-Meier curves for time-to-death by EGFR expression. Subjects with EGFR expression above the median had shorter time-to-death than subjects with lower levels of EGFR (HR=1.69, 95% CI: 1.02, 2.81). EGFR expression showed no correlation with metastasis, age, gender, sarcoma histological classification, or anatomic location. FIG. 2B shows that PLAUR expression significantly correlated with histological classification: levels were below the median in leiomyosarcomas, synovial sarcomas, and dedifferentiated liposarcomas, whereas they were above the median in pleomorphic malignant fibrous histiocytomas (MFH), undifferentiated pleomorphic sarcomas (UPS), and myxofibrosarcomas. FIG. 7 shows that EGFR and PLAUR expression were not correlated. Interestingly, Table 2 shows further statistical analysis that revealed that EGFR expression (p<0.043) and PLAUR expression (p<0.058) were associated with time-to-death. Age, tumor volume, and presence of metastasis also correlated with time-to-death.

TABLE 2
Correlation between patient covariates and death

| Variable | N | HR (95% CI) | | p-value |
|---|---|---|---|---|
| Age (N = 212) | | | | |
| >61 years | 104 | 1.66 | (1, 2.74) | 0.048 |
| ≤61 years | 108 | | | |
| Gender (N = 212) | | | | |
| Male | 95 | 1.07 | (0.65, 1.76) | 0.802 |
| Female | 117 | | | |
| Race = (N = 204) | | | | |
| White | 183 | 0.91 | (0.33, 2.5) | 0.848 |
| Non-White | 21 | | | |
| Tumor Volume (N = 146) | | | | |
| >550 mm³ | 73 | 2.77 | (1.33, 5.79) | 0.007 |
| ≤550 mm³ | 73 | | | |
| Metastasis (N = 134) | | | | |
| Yes | 41 | 2.31 | (1.23, 4.35) | 0.009 |
| No | 93 | | | |
| Tumor Site (N = 212) | | | | |
| Upper abdomen or retroperitoneum | 81 | 1.42 | (0.86, 2.33) | 0.17 |
| Any other location | 131 | | | |
| EGFR (N = 212) | | | | |
| >410 FPKMs | 106 | 1.69 | (1.02, 2.81) | 0.043 |
| ≤410 FPKMs | 106 | | | |
| PLAUR (N = 212) | | | | |
| >757 FPKMs | 106 | 1.63 | (0.98, 2.69) | 0.058 |
| ≤757 FPKMs | 106 | | | |

FIG. 2C shows expression of EGFR and uPAR proteins in human synovial sarcoma TMA. Both proteins were detectable in each of the 54 synovial sarcomas. Table 3 shows more detailed characteristics of these patients and treatments. Neither gene was associated with survival when assessed independently, together, or with other covariates (Table 4).

TABLE 3
Patient information (TMA)

| | | Mean (SD) | |
|---|---|---|---|
| Covariate | Units/Level | or N(%) | |
| Age | Years | 36.9 | (17.1) |
| Sex | Male | 27 | (61.4) |
| | Female | 17 | (38.6) |
| Subtype | Monophasic | 29 | (65.9) |
| | Biphasic | 15 | (34.1) |
| Tumor size | 5 cm or less | 14 | (31.8) |
| | Greater than 5 cm | 29 | (65.9) |
| | Unknown | 1 | (2.3) |
| Lymph Node | No | 43 | (97.7) |
| Involvement | Yes | 1 | (2.3) |
| Metastasis at Diagnosis | No | 41 | (93.2) |
| | Yes | 3 | (6.8) |
| Nodes or Metastasis | No | 40 | (90.9) |
| | Yes | 4 | (9.1) |
| Pre-Surgery Treatment | None | 32 | (72.7) |
| | Chemotherapy | 7 | (15.9) |
| | Radiation | 4 | (9.1) |
| | Chemotherapy and Radiation | 1 | (2.3) |
| Chemotherapy | No | 21 | (47.7) |
| | Yes | 23 | (52.3) |
| Radiation | No | 13 | (29.5) |
| | Yes | 30 | (68.2) |
| | Unknown | 1 | (2.3) |

TABLE 4
Cox regression analysis for time-to-progression

| | | | | | Multivariate Model | | |
|---|---|---|---|---|---|---|---|
| | | | Multivariate Model | | with markers plus other | | |
| | Univariate Model | | with markers only | | covariates | | |
| Covariate | Hazard Ratio | p-value | Hazard Ratio | p-value | Hazard Ratio | | p-value |
| EGFR expression | 0.92 (0.76, 1.11) | 0.369 | 0.86 (0.68, 1.1) | 0.241 | 0.9 | (0.71, 1.14) | 0.401 |
| uPAR expression | 1.02 (0.8, 1.29) | 0.902 | 1.13 (0.85, 1.5) | 0.397 | 0.95 | (0.68, 1.33) | 0.775 |
| Age | | | | | 1 | (0.96, 1.03) | 0.903 |
| Sex (reference = Male) | | | | | 0.64 | (0.19, 2.14) | 0.464 |
| Subtype (reference = | | | | | 1.12 | (0.36, 3.51) | 0.848 |
| monophasic) | | | | | | | |
| Tumor Size (reference = 5 cm | | | | | 3.2 | (0.84, 12.25) | 0.089 |
| or less) | | | | | | | |
| Nodes or Metastasis | | | | | 4.04 | (0.41, 40.26) | 0.234 |
| (reference = no) | | | | | | | |
| Chemotherapy (reference = | | | | | 0.49 | (0.11, 2.11) | 0.336 |
| no) | | | | | | | |
| Radiation (reference = no) | | | | | 0.7 | (0.22, 2.29) | 0.558 |

### Expression of Epidermal Growth Factor Receptor and Urokinase Receptor is Evolutionarily Conserved in Canine HSAs

In order to thoroughly evaluate EGFR and PLAUR expression in canine sarcomas, mRNA expression was evaluated in an independent dataset of 51 canine HSAs by RNAseq (Gorden B H, Kim J H, Sarver A L, et al. Identification of three molecular and functional subtypes in canine hemangiosarcoma through gene expression profiling and progenitor cell characterization. Am J Pathol 2014; 184(4):985-95) and two additional datasets consisting of 31 canine osteosarcomas and 29 canine lymphoma tissue samples (FIGS. 2D, 2E, 2F) (Tonomura N, Elvers I, Thomas R, et al. Genome-wide Association Study Identifies Shared Risk Loci Common to Two Malignancies in Golden Retrievers. PLoS Genet 2015; 11(2):e1004922). Results were similar to those in human sarcomas: expression of both EGFR and PLAUR genes was detectable in all canine sarcomas, with HSA having higher levels of PLAUR mRNA, but equivalent EGFR mRNA. Canine lymphoma samples were included as a negative control and, as expected, expression of both genes was almost undetectable in these samples (<10 FPKMs) as also shown in FIG. 7.

### eBAT is Safe and Effective in Dogs with Spontaneous HSA in a Clinical Setting

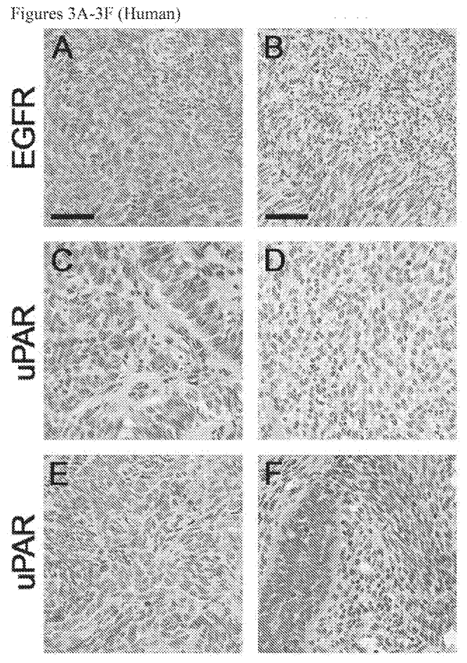

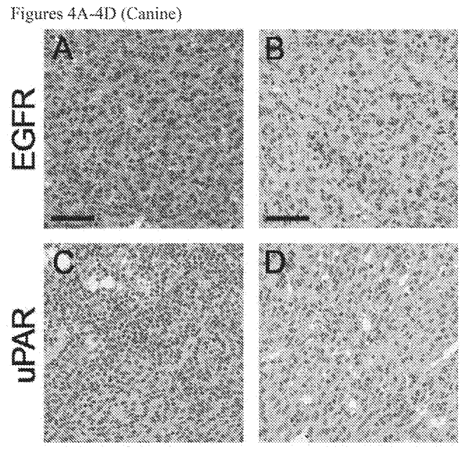

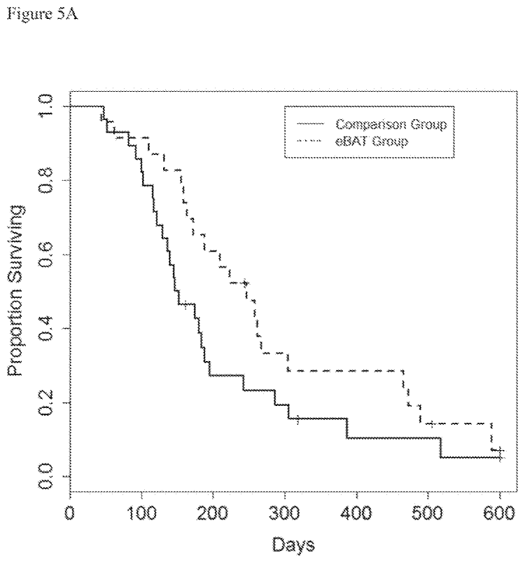

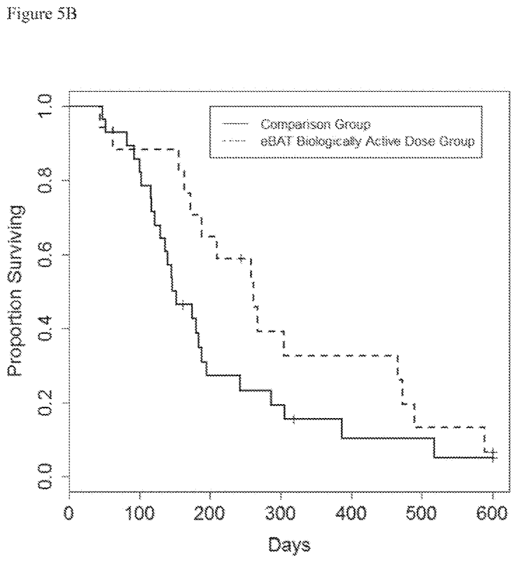

HSA was chosen as a target disease based on its extremely poor prognosis in dogs. Immunostaining of tumor tissues from 15 dogs enrolled in the SRCBST-1 study confirmed that both eBAT targets were expressed at the protein level in all dogs examined replicating the results of immunohistochemical studies in the human synovial sarcoma TMA where both proteins were expressed almost exclusively by tumor cells. FIGS. 3A-3F and 4A-4D shows representative photomicrographs of EGFR and uPAR staining in the canine and human TMAs. Expression of both proteins was variable in non-malignant tissues.

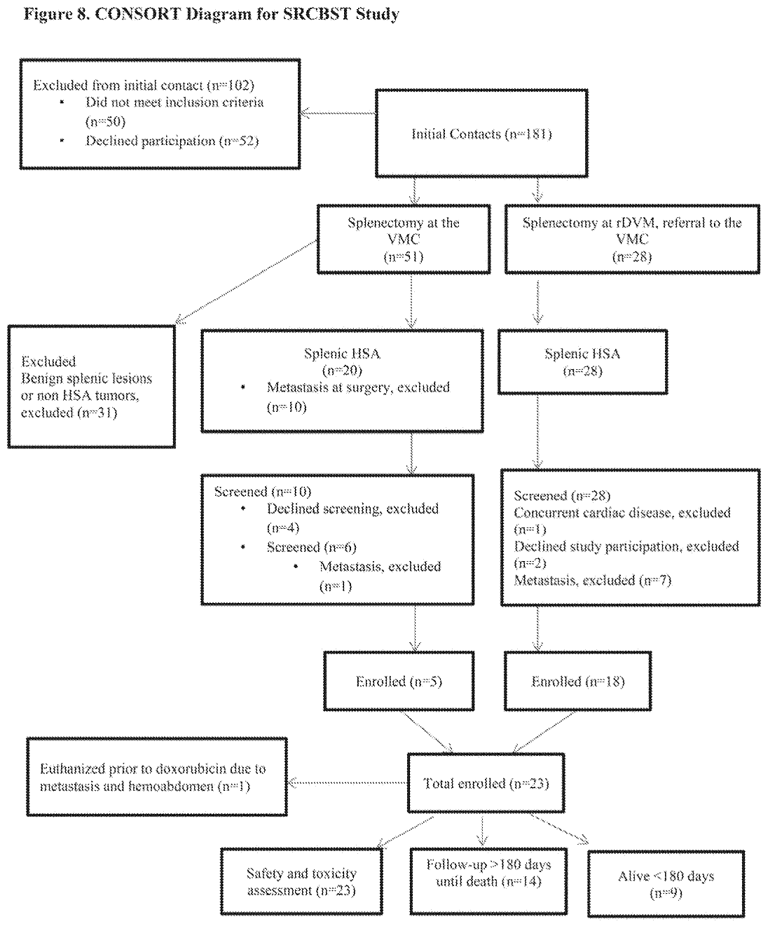

Table 1A summarizes baseline characteristics for all dogs by dose and Table 1B illustrates a treatment timeline for the canine study. A CONSORT diagram showing the flow of study participants is provided in FIG. 8.

eBAT was safe and well tolerated in all dogs with no adverse events (AEs) recorded at 25 μg/kg (dose-1) and only three dogs experiencing AEs at the 50 μg/kg (dose-2). Reversible, hypotensive events were seen in two dogs treated at 100 μg/kg (dose-3). When dog #23 reached the 6-month milestone, interim analysis showed that the study had reached stability at the biologically active dose of 50 μg/kg (dose-2 in the escalation scheme) and was unlikely to change with additional subjects so enrollment was stopped. Based on the favorable trade-off between efficacy and toxicity observed at 50 μg/kg, this dose was identified as the biologically active dose, and was used for all subsequent cohorts.

Median survival for the 23 dogs treated with adjuvant eBAT (eBAT group) was 8.1 months (FIG. 4A) compared to 4.9 months for the Comparison group of dogs treated with standard of care alone. Median survival was 8.6 months for the 17 dogs treated at the biologically active dose (FIG. 4B). Overall, six-month survival rates were 65.2%, and 70.6%, and 38.7%, for the eBAT group, the group treated at the biologically active dose, and the Comparison group, respectively.

Average time from splenectomy to initiation of chemotherapy was shorter in the Comparison group (20.8 days) than in the eBAT group (43.7 days) or the group treated at the biologically active dose (46.2 days). Six (26%) of 22 enrolled dogs and five of 16 (29%) treated at the biologically active dose exceeded 1-year survival; one dog had not reached the 1-year follow-up mark and was still alive when the study closed. Five of the remaining 22 dogs (22.7%) had no evidence of relapse by staging with survival >450 days when enrollment stopped. Detectable levels of eBAT were achieved in the systemic circulation of dogs treated by intravenous infusion (not shown).

### eBAT Shows Limited Toxicity In Vivo

For our companion canine study, the estimated probabilities of AEs by dose are shown in Table 5A, and specific information regarding AEs is shown in Table 5B. Grade 1-3 toxicities associated with subsequent doxorubicin chemotherapy were predictable and limited to 12 dogs in total. No dogs experienced cutaneous, ocular, gastrointestinal toxicity or laboratory abnormalities that have been previously associated with EGFR targeted therapies in humans (Funakoshi T, Latif A, Galsky M D. Safety and efficacy of addition of VEGFR and EGFR-family oral small-molecule tyrosine kinase inhibitors to cytotoxic chemotherapy in solid cancers: a systematic review and meta-analysis of randomized controlled trials. Cancer Treat Rev 2014; 40(5):636-47). Necropsy was performed in 2 of 23 dogs and showed no evidence of chronic changes attributable to eBAT. Both of these dogs died due to progressive HSA.

Since other studies have shown that EGFR-targeted therapies are associated with significant dose-limiting cutaneous and gastrointestinal toxicities (Funakoshi T, Latif A, Galsky M D. Safety and efficacy of addition of VEGFR and EGFR-family oral small-molecule tyrosine kinase inhibitors to cytotoxic chemotherapy in solid cancers: a systematic review and meta-analysis of randomized controlled trials. Cancer Treat Rev 2014; 40(5):636-47; Launay-Vacher V, Aapro M, De Castro G, Jr., et al. Renal effects of molecular targeted therapies in oncology: a review by the Cancer and the Kidney International Network (C-KIN). Ann Oncol 2015; 26(8):1677-84), the safety of eBAT versus EGF-toxin alone in normal C57BL/6 mice was further examined. Maximum tolerated doses were established for monospecific EGF-toxin given alone (20 μg/kg), monospecific uPA-toxin given alone (40 μg/kg) and both drugs administered jointly (40 μg/kg); most deaths occurred within 7 days post-treatment. There were no deaths or gross toxicities in mice receiving up to 160 μg/kg of eBAT (Table 5C). As previously stated, we selected a conservative starting dose of 25 μg/kg for clinical testing in dogs with spontaneous HSA.

Tables 5A-5C: Adverse Events (AEs) for Dogs in the SRCBST study and Mice Treated with eBAT

### A. Summary of AEs Including the Empirical and Model-Based Estimated Rate by Treatment Group

| | | | AE Rate - empirical | | AE Rate - from model | |
|---|---|---|---|---|---|---|
| Dose Level | N | AEs* | (95% CI) | | (95% CI) | |
| 1 (25 ug/kg) | 3 | 0 | 0% | (0%, 70.8%) | 10.1% | (0.3%, 31.9%) |
| 2 (50 ug/kg) | 17 | 3 | 17.6% | (3.8%, 43.4%) | 19.5% | (6.6%, 37.7%) |
| 3 (100 ug/kg) | 3 | 2 | 66.7% | (9.4%, 99.2%) | 44.4% | (10.3%, 90.6%) |

*Total count of dogs experiencing AEs (not total number of AEs)

### B. Description of AEs in Individual Dogs, Management, and Outcome

| Dog ID and | Dose | | | |
|---|---|---|---|---|
| Breed | Level | AEs | Management | Outcome |
| MN11 | 2 | Grade 3 ALT elevation after 1^{st} infusion | Second eBAT infusion delayed one week | Full |
| Cairn Terrier | | | | recovery |
| | | Hypotensive event* during 2^{nd} infusion | IV fluid bolus | Full |
| | | | 3^{rd} eBAT infusion not administered | recovery |
| MN17 | 2 | Hypotensive event followed by a seizure | IV fluid bolus, infusion restated 45 minutes later | Full |
| Labrador | | during 1^{st} infusion | with no complications | recovery |
| Retriever | | | | |
| MN22 | 2 | Grade 2 ALT elevation after 1^{st} infusion | Monitoring | Full |
| Rat Terrier | | | | recovery |
| MN07 | 3 | Hypotensive event at the end of 3^{rd} | IV fluid bolus | Full |
| Newfoundland | | infusion | | recovery |
| MN09 | 3 | Hypotensive event during second infusion | IV fluid bolus, infusion not restarted | Full |
| Goldendoodle | | | | recovery |

*Hypotensive events noted in 4 dogs were characterized by mean arterial pressure <60 mmHg, hind limb weakness, pale mucous membranes, weak femoral pulses, and a single vomiting episode in one dog. All other dogs had no adverse events.

C. Summary of Death Events in Normal Mice Treated with Ligand Specific Toxins

| | Observed Deaths (%) | | | | | | | | |
|---|---|---|---|---|---|---|---|---|---|
| | Dose (μg/kg) | | | | | | | | |
| Treatment | 10 | 20 | | 40 | | 80 | | 160 | |
| Monospecific | 0/8 (0) | 2/8 | (25) | 6/8 | (75) | 8/8 | (100) | 8/8 | (100) |
| EGF-toxin | | | | | | | | | |
| Monospecific | 0/8 (0) | 0/8 | (0) | 2/8 | (25) | 8/8 | (100) | 8/8 | (100) |
| uPA-toxin, | | | | | | | | | |
| Monospecific | 0/7 (0) | 1/7 | (14) | 2/7 | (29) | 7/7 | (100) | 7/7 | (100) |
| EGF-toxin + | | | | | | | | | |
| monospecific | | | | | | | | | |
| uPA-toxin | | | | | | | | | |
| eBAT | 0/8 (0) | 0/8 | (0) | 0/8 | (0) | 0/8 | (0) | 0/8 | (0) |

### Anti-eBAT Entibody Responses are Sporadic and do not Interfere with Clinical Responses

eBAT contains a bacterial toxin, so immunogenicity was expected and considered as a potential barrier to bioactivity. Samples for NA measurement were available for all dogs at baseline, 19/23 dogs on Day 8, and 7/23 dogs on Day 21.

Dogs in which we could detect drug in the circulation on Day 1 had significantly better survival (p=0.003) than dogs in which drug was undetectable. Drug was detectable on Day 1 in 4 of 9 dogs with no evidence of antibody at baseline or following eBAT administration, 7 of 9 dogs with antibody formation after eBAT treatment, and 1 of 4 dogs with pre-existing antibody (this dog was treated at the highest dose). No associations were found between survival and detectable drug at days 5 or 6 (p=0.526), AUC at day 1 (p=0.64), AUC at day 5 or 6 (p=0.82) or the presence of neutralizing antibodies (p=0.712).

### Discussion

The major contributions of this study were the following: 1) first-time evaluation of a potent bispecific, anti-angiogenic targeted toxin in an “ontarget” large animal sarcoma model demonstrating potent anti-sarcoma activity and long-term survival, 2) description of an EGFR-targeted therapy that is surprisingly well-tolerated, and 3) findings supporting our belief that bispecificity targeting reduces toxicity risks associated with EGFR targeting.

eBAT was tested in a model of canine HSA, using an adaptive study design in the minimal residual disease setting. A biologically active dose was identified that was safe and effective. None of the treated dogs experienced signs of capillary leak syndrome, the toxicity of greatest concern for immunotoxins (Bachanova V, Frankel A E, Cao Q, et al. Phase I study of a bispecific ligand-directed toxin targeting CD22 and CD19 (DT2219) for refractory B-cell malignancies. Clin Cancer Res 2015; 21(6):1267-72; Smallshaw J E, Ghetie V, Rizo J, et al. Genetic engineering of an immunotoxin to eliminate pulmonary vascular leak in mice. Nat Biotechnol 2003; 21(4):387-91). Furthermore, the lack of adverse events similar to those caused by EGFR-targeted therapies (Funakoshi T, Latif A, Galsky M D. Safety and efficacy of addition of VEGFR and EGFR-family oral small-molecule tyrosine kinase inhibitors to cytotoxic chemotherapy in solid cancers: a systematic review and meta-analysis of randomized controlled trials. Cancer Treat Rev 2014; 40(5):636-47; Launay-Vacher V, Aapro M, De Castro G, Jr., et al. Renal effects of molecular targeted therapies in oncology: a review by the Cancer and the Kidney International Network (C-KIN). Ann Oncol 2015; 26(8):1677-84) suggests that the addition of the uPAR-directed ligand enhances targeting specificity to tumors, leading to diminished toxicity, consistent with our mouse data.

Bispecificity is one unique aspect of eBAT, as this may permit reactivity with a wider range of cell surface markers, enhancing the ability to kill resistant tumor cell outliers. In the case of eBAT, studies showed an ability to simultaneously target uPAR on human vascular endothelial cells (HUVEC cells) and EGFR on tumor cells (Tsai A K, Oh S, Chen H, et al. A novel bispecific ligand-directed toxin designed to simultaneously target EGFR on human glioblastoma cells and uPAR on tumor neovasculature. J Neurooncol 2011; 103(2):255-66). It is believed that bispecificity contributed to the notable clinical effect. The results are further strengthened by the design that allowed dose finding to be guided by safety and 6-month survival (Koopmeiners J S, Modiano J. A Bayesian adaptive Phase I-II clinical trial for evaluating efficacy and toxicity with delayed outcomes. Clin Trials 2014; 11(1):38-48), in turn allowing us to identify a biologically active dose without having to establish a maximum tolerated dose (MTD). Indeed, the data suggest the biologically active dose is lower than the MTD. The striking clinical results could be also due to testing of the drug in the minimal residual disease setting, which is a unique opportunity afforded by the canine model and is in contrast to other studies of immunotoxins in humans, where bulky, refractory, heavily pre-treated tumor loads exceed the capabilities of the test article.

Six of seven dogs had NAs on day 21, suggesting that the use of a deimmunized toxin was justified (Onda M, Nagata S, FitzGerald D J, et al. Characterization of the B cell epitopes associated with a truncated form of Pseudomonas exotoxin (PE38) used to make immunotoxins for the treatment of cancer patients. J Immunol 2006; 177(12):8822-34.). Nonetheless, the presence of NAs was not associated with survival outcomes, and there was no correlation between NAs and the dose of eBAT received or the drug PK. These findings were similar to other studies with targeted toxin where antitoxin antibody titers did not correlate with antitumor activity (Frankel A E, Woo J H, Ahn C, Foss F M, Duvic M, Neville P H, Neville D M. Resimmune, an anti-CD3c recombinant immunotoxin, induces durable remissions in patients with cutaneous T-cell lymphoma. Haematologica (2015); June; 100(6): 794-800). The results exceeded expectations for outcome of dogs with stage-I or stage-II HSA based on the historical data and on other published data from comparable populations treated with the standard of care (Thamm DH. Miscellaneous tumors. In: Withrow S J, Vail D M, Page R L, (eds). Withrow and MacEwen's Small Animal Clinical Oncology. 5th ed. St. Louis: Elsevier; 2013, 679-688; Clifford C A, Mackin A J, Henry C J. Treatment of canine hemangiosarcoma: 2000 and beyond. J Vet Intern Med 2000; 14(5):479-85). In fact, dogs receiving eBAT had longer survival times than dogs treated with any other contemporary experimental therapy (Thamm D H. Miscellaneous tumors. In: Withrow S J, Vail D M, Page R L, (eds). Withrow and MacEwen's Small Animal Clinical Oncology. 5th ed. St. Louis: Elsevier; 2013, 679-688; Clifford C A, Mackin A J, Henry C J. Treatment of canine hemangiosarcoma: 2000 and beyond. J Vet Intern Med 2000; 14(5):479-85; Lana S, U'Ren L, Plaza S, et al. Continuous low-dose oral chemotherapy for adjuvant therapy of splenic hemangiosarcoma in dogs. J Vet Intern Med 2007; 21(4):764-9; U'Ren L W, Biller B J, Elmslie R E, et al. Evaluation of a novel tumor vaccine in dogs with hemangiosarcoma. J Vet Intern Med 2007; 21(1):113-20; Chon E, McCartan L, Kubicek L N, et al. Safety evaluation of combination toceranib phosphate (Palladia(R)) and piroxicam in tumour-bearing dogs (excluding mast cell tumours): a phase I dose-finding study. Vet Comp Oncol 2012; 10(3):184-93). The one-year survival for dogs treated at the biologically active dose was almost 40% and the proportion of dogs living 6 months or longer nearly doubled compared to a comparison population. Five dogs were considered cured from this fatal disease.

It is intriguing that time to initiation of chemotherapy was longer in dogs treated with eBAT than in the Comparison group. It is generally assumed that a shorter time to initiation of chemotherapy would produce more favorable outcomes, but survival was longer in dogs treated with eBAT even though chemotherapy was delayed. It is unlikely that the variability in chemotherapy protocols used in the Comparison group had an impact on survival since, historically, single agent doxorubicin and combination protocols are equally effective (Thamm D H. Miscellaneous tumors. In: Withrow S J, Vail D M, Page R L, (eds). Withrow and MacEwen's Small Animal Clinical Oncology. 5th ed. St. Louis: Elsevier; 2013, 679-688; Clifford C A, Mackin A J, Henry C J. Treatment of canine hemangiosarcoma: 2000 and beyond. J Vet Intern Med 2000; 14(5):479-85). Because metastatic disease occurred in about half of the dogs, it is possible that repeat cycles could prolong remissions as has been shown in studies with targeted Pseudomonas exotoxin in humans (Bachanova V, Frankel A E, Cao Q, et al. Phase I study of a bispecific ligand-directed toxin targeting CD22 and CD19 (DT2219) for refractory B-cell malignancies. Clin Cancer Res 2015; 21(6):1267-72; Chon E, McCartan L, Kubicek L N, et al. Safety evaluation of combination toceranib phosphate (Palladia(R)) and piroxicam in tumour-bearing dogs (excluding mast cell tumours): a phase I dose-finding study. Vet Comp Oncol 2012; 10(3):184-93; Kreitman R J, Tallman M S, Robak T, et al. Phase I trial of anti-CD22 recombinant immunotoxin moxetumomab pasudotox (CAT-8015 or HA22) in patients with hairy cell leukemia. J Clin Oncol 2012; 30(15):1822-8).

The mechanism of action of eBAT remains to be fully elucidated. In this study, both eBAT targets were expressed in human sarcoma samples. Thus, current TCGA findings and synovial sarcoma TMA analysis support other reports in the literature (Albritton K H, Randall R L. Prospects for targeted therapy of synovial sarcoma. J Pediatr Hematol Oncol 2005; 27(4):219-22; Yang J L, Hannan M T, Russell P J, et al. Expression of HER1/EGFR protein in human soft tissue sarcomas. Eur J Surg Oncol 2006; 32(4):466-8; Tschoep K, Kohlmann A, Schlemmer M, et al. Gene expression profiling in sarcomas. Crit Rev Oncol Hematol 2007; 63(2):111-24; Benassi M S, Ponticelli F, Azzoni E, et al. Altered expression of urokinase-type plasminogen activator and plasminogen activator inhibitor in high-risk soft tissue sarcomas. Histol Histopathol 2007; 22(9):1017-24) regarding EGFR and PLAUR expression. A recent study confirmed that uPAR was expressed in 100% (57/57) of canine HSAs tested, but only in 30% (8/26) of hemangioma samples (Anwar S, Yanai T, Sakai H. Immunohistochemical Detection of Urokinase Plasminogen Activator and Urokinase Plasminogen Activator Receptor in Canine Vascular Endothelial Tumours. J Comp Pathol 2015; 153(4):278-82). Here, expression of both targets in canine HSA samples and expression was present in the tumor cells and/or in the tumor microenvironment, but they also were present in normal tissues. Taken together, the expression data indicate that these markers are excellent targets and eBAT may be highly effective in sarcoma intervention. Furthermore, the data suggest that the excellent safety profile could be due to a unique reactivity with tumor cells, although it also could be due to the extremely low dose required to control or ablate the mass of malignant cells present in the minimal residual disease setting. However, we cannot exclude the possibility that eBAT makes the microenvironment inhospitable for tumor formation. The consistently high expression of uPAR in tumor-associated mononuclear inflammatory cells also raises the possibility that eBAT acts through a primary immune mechanism by eliminating or attenuating this cellular compartment, which in turn removes a strong impetus for tumor formation and/or tumor progression (Gorden B H, Kim J H, Sarver A L, et al. Identification of three molecular and functional subtypes in canine hemangiosarcoma through gene expression profiling and progenitor cell characterization. Am J Pathol 2014; 184(4):985-95; Kim J H, Frantz A M, Anderson K L, et al. Interleukin-8 promotes canine hemangiosarcoma growth by regulating the tumor microenvironment. Exp Cell Res 2014; 323(1):155-64; Tamburini B A, Phang T L, Fosmire S P, et al. Gene expression profiling identifies inflammation and angiogenesis as distinguishing features of canine hemangiosarcoma. BMC Cancer 2010; 10:619). The fact that EGF4KDEL was not as effective as EGFATFKDEL (eBAT) in vitro suggests that simultaneously targeting EGFR and uPAR may be essential for optimal efficacy of this drug.

In conclusion, it was demonstrated that eBAT is safe, and that the addition of a uPAR directed ligand to the EGFR targeting molecule abrogated the dose-limiting cutaneous, ocular, and gastrointestinal toxicities, or hypomagnesemia generally associated with EGFR targeting. We also showed that eBAT has biological activity in a highly metastatic, incurable canine sarcoma that carries many similarities with human disease. The strategy is not aimed at modulating EGF or uPA-dependent pathways, since neither EGFR nor uPAR appear to act as drivers of tumor progression. Rather the proteins act as “bait” for a ligand-targeted cytotoxic therapy. Given that the targets are invariably expressed in human sarcomas, our data provides a strong rationale for translation of eBAT in the treatment of human sarcomas and potentially other EGFR and uPAR-expressing tumors.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a subject having a tumor that expresses EGFR and/or uPAR, even if at low levels comprising:
administering intravenous fluids to the subject at a rate of about 0.1 to 1 ml/kg/hr for about 10 to 60 minutes, and
systemically administering a therapeutic composition at a dosage regime of 25 to 100 μg/kg, wherein the therapeutic composition is administered three times in a one-week period.

2-21. (canceled)

2. A method of treating a subject having a tumor that expresses EGFR and/or uPAR, even if at low levels that would otherwise be at risk for toxicity related to EGFR and/or uPAR targeted therapies, comprising:
administering intravenous fluids to the subject at a rate of about 0.1 to 1 ml/kg/hr for about 10 to 60 minutes, and
systemically administering a therapeutic composition at a dosage regime of 25 to 100 μg/kg, wherein the therapeutic composition is administered three times in a one-week period.

3. The method of claim 1, wherein the level of EGFR is a low level of EGFR.

4. The method of claim 1, wherein the level of uPAR is a low level of uPAR.

5. The method of any one of claims 1, wherein the tumor is a carcinoma or a sarcoma.

6. The method of claim 5, wherein the sarcoma is a hemangiosarcoma (HSA) or an angiosarcoma.

7. The method of claim 5, wherein the subject is a human and the tumor is a sarcoma.

8. The method of claim 5, wherein the subject is a dog and the tumor is an HSA.

9. A method of preventing hemangiosarcoma (HSA) in a dog predisposed to developing HSA or angiosarcoma in a human predisposed to developing angiosarcoma comprising:
(a) administering intravenous fluids to the subject at a rate of about 0.1 to 1 ml/kg/hr for about 10 to 60 minutes, and
(b) systemically administering a therapeutic composition at a dosage regime of 25 to 100 μg/kg, wherein the therapeutic composition is administered three times in a one-week period.

10. The method of any one of claims 1, wherein step (b) is repeated one or more times.

11. The method of any one of claims 1, further comprising (c) administering chemotherapy.

12. The method of claim 11, further comprising (d) repeating step (b).

13. The method of any one of claims 1, wherein the therapeutic composition is eBAT, wherein eBAT is EGFATF-KDEL, EGFATF-KDEL-mut7, and/or EGFuPA-toxin.

14. The method of any one of claims 1, wherein the therapeutic composition comprises EGFATFKDEL mut7.

15. The method of any one of claims 1, wherein the therapeutic composition comprises EGFKDEL and ATFKDEL, wherein EGFKDEL and ATFKDEL are administered separately, simultaneously or sequentially.

16. The method of any one of claims 1, wherein the dosage of the therapeutic composition is about 50 μg/kg.

17. The method of any one of claims 1, wherein the therapeutic composition is administered by means of a slow IV push.

18. The method of any one of claims 1, wherein steps (a) and (b) are repeated about one to three weeks after initial treatment.

19. The method of any one of claims 1, wherein the systemic administration is by means of intravenous, intraperitoneal or subcutaneous administration.

20. The method of any one of claims 1, wherein the administration is daily.

21. The method of any one of claims 1, wherein the administration the administration is for a duration of at least six months.
